(19) United States
(12) Patent Application Publication
(10) Pub. No.: US 2006/0173063 A1
(43) Pub. Date: Aug. 3, 2006

(54) Exo-(t-butyl 2R(+))-2-amino-7-azabicyclo[2.2.1]heptane-7-carboxylate, intermediates, and process to prepare and isolate them

(75) Inventors: Thomas Lee Fevig, Wildwood, MO (US); Tizah Evelyn Anjeh, O'Fallon, MO (US); Jon Philip Lawson, Glencoe, MO (US); Daniel P. Walker, Mystic, CT (US); Donn Gregory Wishka, Mystic, CT (US)

Correspondence Address: PFIZER INC, 150 EAST 42ND STREET, 5TH FLOOR - STOP 49, NEW YORK, NY, 10017-5612, US

(73) Assignee: Pfizer Inc.

(21) Appl. No.: 10/782,625

(22) Filed: Feb. 19, 2004

Related U.S. Application Data
(60) Provisional application No. 60/449,242, filed on Feb. 21, 2003.

(51) Int. Cl. C07D 487/02; A61K 31/407

(52) U.S. Cl. 514/412; 548/453

(57) ABSTRACT

The present invention relates to exo-(t-butyl 2R(+))-2-amino-7-azabicyclo[2.2.1]heptane-7-carboxylate (formula 1) a novel compound, and the process for the preparation thereof, and novel intermediates therein.

### CROSS REFERENCE TO RELATED APPLICATIONS

This application claims the benefit of U.S. provisional application Ser. No. 60/449,242 filed on 21 Feb. 2003, under 35 USC 119(e)(i), which is incorporated herein by reference in its entirety.

### FIELD OF INVENTION

The present invention relates to exo-(t-butyl 2R(+))-2-amino-7-azabicyclo[2.2.1]heptane-7-carboxylate (formula 1) a novel compound, and the process for the preparation thereof, and novel intermediates therein.

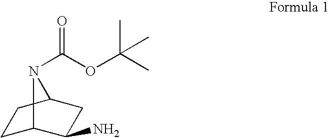

### BACKGROUND OF THE INVENTION

Exo-(t-butyl 2R(+))-2-amino-7-azabicyclo[2.2.1]heptane-7-carboxylate is useful for the preparation of compounds or medicaments useful for the treatment of diseases, including, but not limited to, diseases of the central nervous system and other diseases where a mammal would receive symptomatic relief from the activation of alpha 7 nicotinic acetylcholine receptors (α7 nAChRs). Nicotinic acetylcholine receptors (nAChRs) play a large role in central nervous system (CNS) activity and in different tissues throughout the body. There are several types of nAChRs, and each one appears to have a different role. Some nicotinic receptors regulate CNS function (they are known to be involved in functions, including, but not limited to, cognition, learning, mood, emotion, and neuroprotection); some regulate pain, inflammation, cancer, and diabetes by controlling tumor necrosis factor alpha (TNF-α); and some regulate vascular angiogenesis (for example, the binding of nicotine to the alpha-7 nAChR stimulates DNA synthesis and proliferation of vascular endothelial cells in vitro (Villablanca, A. C., 1998, J. Appl. Physiol., 84(6):2089-2098) and induces angiogenesis in vivo (Heeschen C., et al. 2002, J. Clin. Invest., 110:527-535; Heeschen, C., et al. 2001, Nature Medicine, 7(7): 833-839)). Nicotine affects all such receptors, and has a variety of activities. Unfortunately, not all of the activities are desirable. In fact, undesirable properties of nicotine include its addictive nature and the low ratio between efficacy and safety.

Cell surface receptors are, in general, excellent and validated drug targets. nAChRs comprise a large family of ligand-gated ion channels that control neuronal activity and brain function. These receptors have a pentameric structure. In mammals, this gene family is composed of nine alpha and four beta subunits that co-assemble to form multiple subtypes of receptors that have a distinctive pharmacology. Acetylcholine is the endogenous regulator of all of the subtypes, while nicotine non-selectively activates all nAChRs.

The α7 nAChR is one receptor system that has proved to be a difficult target for testing. Native α7 nAChR is not routinely able to be stably expressed in most mammalian cell lines (Cooper and Millar, J. Neurochem., 1997, 68(5):2140-51). Another feature that makes functional assays of α7 nAChR challenging is that the receptor is rapidly (100 milliseconds) inactivated. This rapid inactivation greatly limits the functional assays that can be used to measure channel activity.

Agonists of the α7 nAChR are assayed using a cell-based, calcium flux assay on FLIPR. SHEP-1 cells expressing a novel, mutated form of the α7 nAChR that permitted stable cell surface expression were used for these assays. The details of the mutated form of the α7 nAChR are described in WO 00/73431.

Exo-(t-butyl 2R(+))-2-amino-7-azabicyclo[2.2.1]heptane-7-carboxylate is a precursor for making full α7 nAChRs agonists. The present invention provides a safer and efficient route for the preparation of this key intermediate having absolute stereochemistry at C-2 of the azabicyclic ring system of the 2-amino-7-azabicyclo[2.2.1]heptane intermediate. Synthetic approaches to this ring system have been summarized in a recent review (Chen, Z. and Trudell, M. L., Chem. Rev., 1996, 96, 1179). In almost all of these approaches, the ultimate target was the naturally occurring alkaloid, epibatidine. However, exo-(t-butyl 2R(+))-2-amino-7-azabicyclo[2.2.1]heptane-7-carboxylate has not been prepared to our knowledge.

U.S. Pat. No. 6,255,490 discloses 7-azabicyclo[2.2.1]-heptane and -heptene derivatives as cholinergic receptor ligands.

U.S. Pat. No. 6,117,889 discloses 7-azabicyclo[2.2.1]-heptane and -heptene derivatives as analgesics and anti-inflammatory agents.

U.S. Pat. No. 6,060,473 discloses 7-azabicyclo[2.2.1]-heptane and -heptene derivatives as cholinergic receptor ligands.

U.S. Pat. No. 6,054,464 discloses azabicyclic esters of carbamic acids useful in therapy, especially in the treatment or prophylaxis of psychotic disorders and intellectual impairment disorders, as well as intermediates and use of intermediates in synthesis.

U.S. Pat. No. 5,817,679 discloses 7-azabicyclo[2.2.1]-heptane and -heptene derivatives as cholinergic receptor ligands.

GB 1,167,688 discloses dealkylation of cyclic N-alkyl derivatives.

In Chen, Z. and Trudell, M. L., Chem. Rev., 1996, 96, 1179, the chemistry of 7-azabicyclo[2.2.1]hepta-2,5-dienes, 7-azabicyclo[2.2.1]hepta-2-enes and 7-azabicyclo[2.2.1]heptanes is discussed.

In Fletcher, et al., J. Chem. Soc. Commun., 1993, 15, 1216-1218, the synthesis of (+)- and (−)-Epibatidine is discussed.

In Grunewald, G. L. et al., J. Med. Chem., 1988, 31, 433, conformational and steric aspects of the inhibition of phenylethanolamine N-methyltransferase by benzylamines is discussed.

In Salvatore, R. N. et al., J. Org. Chem., 2001, 66, 1035, the efficient carbamate synthesis via a three-component coupling of an amine, CO₂, and alkyl halides in the presence of Cs₂CO₃ and tretrabutylamonium iodide is discussed.

In Olivo and Hemenway, J. Org. Chem., 1999, 64(24), 8968-8969, the total synthesis of (+/−)Epibatidine using a biocatalytic approach is discussed.

In Zhang, C. and Trudell, M. L., J. Org. Chem., 1996, 61, 7189, a short and efficient total synthesis of (+/−)-Epibatidine is discussed.

In Bai, D.; Xu, R. et al., J. Org. Chem., 1996, 61, 4600, synthesis of (+/−)-Epibatidine and its analogs is discussed.

In Fletcher, et al., J. Org. Chem., 1994 59(7), 1771-1778, total synthesis and determination of the absolute configuration of Epibatidine is discussed.

In House, H. O. et al., J. Org. Chem., 1968, 34, 2324, preparation of trimethylsilyl enol ethers is discussed.

In Cheng, J. and Trudell, M. L, Org. Lett., 2001, 3(9), 1371-1374, the synthesis of N-heteroaryl-7-azabicyclo[2.2.1]heptane derivatives via palladium-bisimidazol-2-ylidene complex catalyzed amination reactions is discussed.

In Ramanaiah, K. V C. V., et al., Org. Lett., 1999 1(9) 1439-1441, the synthesis and sterochemical assignment of exo- and endo-7-methyl-7-azabicyclo[2.2.1]heptan-2-ol is discussed.

In Kende, A. S., Org. Reactions, 1960, 11, 261, the Favorskii rearrangement of haloketones is discussed.

In Cabanal-Duvillard, I., et al., Tetrahedron, 2000, 56, 3763-3769; a formal asymmetric synthesis of (−)-Epibatidine using a highly diastereoselective hetero Diels-Alder reaction is discussed.

In Karstens, Willem F. J., et al., Tetrahedron. Lett., 1999, 40, 8629-8632, the application of an organozinc reagent derived from (S)-Pyroglutamic acid is discussed for a formal synthesis of Epibatidine.

In Cabanal-Duvillard, I., et al., Tetrahedron. Lett., 1998, 39(29), 5181-5184, the expeditious formal synthesis of (+/−)-Epibatidine using diastereoselective bromohydroxylation of aminocyclohexene derivatives is discussed.

In Bajwa, J. S., Tetrahedron. Lett., 1992, 33, 2955, a one-pot transformation of benzyl carbamates into t-butyl carbamates is discussed.

### SUMMARY OF INVENTION

The present invention provides a safe and efficient route for the preparation of exo-(t-butyl 2R(+))-2-amino-7-azabicyclo[2.2.1]heptane-7-carboxylate, formula 1, a key intermediate having absolute 1S, 2R, 4R or exo-2R stereochemistry on the azabicyclic ring system. The terms exo and endo are stereochemical prefixes that describe the relative configuration of a substituent on a bridge (not a bridgehead) of a bicyclic system. If a substituent is oriented toward the larger of the other bridges, it is endo. If a substituent is oriented toward the smaller bridge, it is exo. Depending on the substitution on the carbon atoms, the endo and exo orientations can give rise to different stereoisomers. For instance, in Formula 1, when carbons 1 and 4 are substituted with hydrogen and carbon 2 is bonded to a nitrogen-containing species, the endo orientation gives rise to the possibility of a pair of enantiomers: either the 1S, 2S, 4R isomer or its enantiomer, the 1R, 2R, 4S isomer. Likewise, the exo orientation gives rise to the possibility of another pair of stereoisomers which are diastereomeric and C-2 epimeric with respect to the endo isomers: either the 1R, 2S, 4S isomer or its enantiomer, the 1S, 2R, 4R isomer. The compounds of the present invention are 1S, 2R, 4R, or exo-2(R).

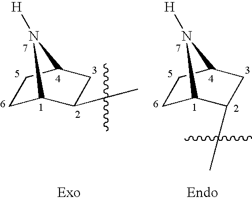

Compounds having the azabicyclo[2.2.1]heptane skeleton have been described; synthetic approaches to this ring system have been summarized in a recent review (Chen, Z. and Trudell, M. L., Chem. Rev., 1996, 96, 1179). In almost all of these approaches, the ultimate target was the naturally occurring alkaloid, epibatidine.

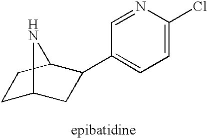

Careful examination of these approaches revealed that most of them were unsuitable for adaptation to large-scale preparation of formula 1. Many of the routes entailed an excessive number of steps, or used raw materials that were hazardous or not readily available. Others proceeded through intermediates that were hazardous, or contained functionality detrimental to the synthesis of 1.

A recent synthesis of epibatidine reported by Bai et al (Bai, D.; Xu, R. et al., J. Org. Chem., 1996, 61, 4600), held potential for adaptation to a synthesis of formula 1. The reference reported that epibatidine could be obtained from tropinone using a Favorskii rearrangement to obtain the 2.2.1 azabicyclic ring system in epibatidine. This synthesis used relatively inexpensive, non-hazardous and readily available raw materials, and went through an intermediate that could be a precursor to 1:

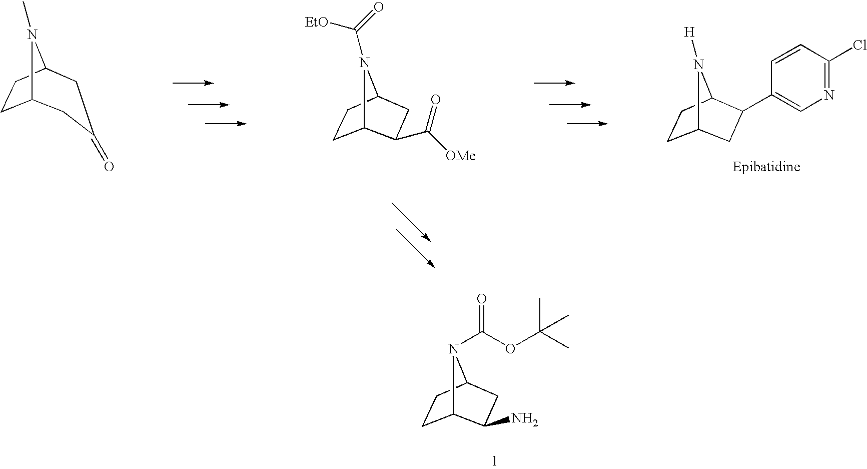

Our attempts to apply the chemistry reported by Bai in the process to give formula 1 had several problems. For example, the ethyl carbamate group used in the Bai synthesis could not be removed in the presence of the functionality required for the synthesis of formula 1. Also, upon close examination, the conditions used by Bai in the key Favorskii rearrangement step were found to give an approximate 3/1 mixture of diastereoisomers, as well as partial carbamate interchange. This rendered the procedure unscalable to make formula 1. Surprisingly, we were able to overcome these difficulties to find a novel, scalable, safe, and efficient synthesis of 1. See Scheme 1. First, we found that the phenyl carbamate provided an intermediate that is stable to the bromination conditions and is easily removed later. Also, the conditions used for the Favorskii rearrangement were modified so as to produce an easily manipulated benzyl carbamate by using sodium benzyloxide as the base in the reaction. Surprisingly, carbamate interchange allowed this to occur. Moreover, by using benzyl alcohol, a dramatic improvement in the stereoselectivity of the reaction is obtained. The improved selectivity allows an isomerization and/or a purification step to be avoided, and simplified downstream processing. Another aspect of the present invention is the preparation of 1 according to the process as outlined in Scheme 1.

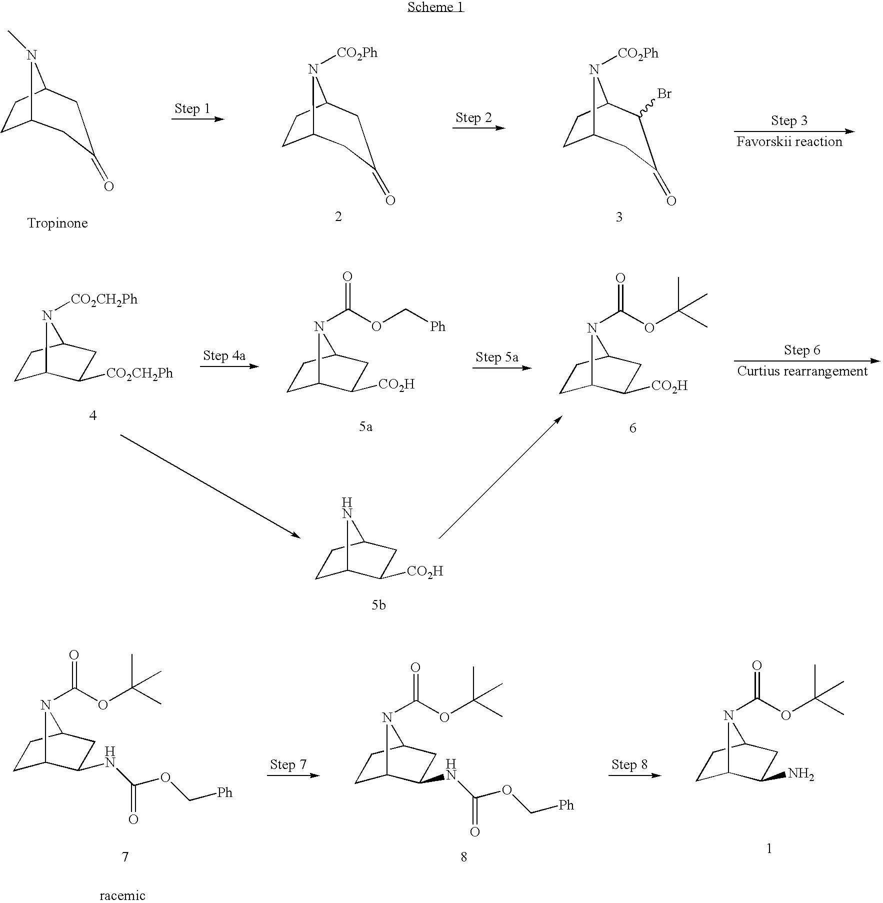

Application of these solutions led to an efficient, safe and scalable synthesis of dibenzyl 7-azabicyclo[2.2.1]heptane-2,7-dicarboxylate (4). This new compound could be converted into 1 by the sequence of reactions shown. Because of the very high selectivity of the Favorskii reaction, 7-(tert-butoxycarbonyl)-7-azabicyclo[2.2.1]heptane-2-carboxylic acid (6) could ultimately be obtained as a single diastereoisomer (exo, as shown). This was done by removing the protecting groups from 4 and installing the required t-butyl carbamate group to give carboxylic acid 6. The amino group is introduced by taking advantage of the Curtius rearrangement, which is known to proceed with retention of stereochemistry. The product of the Curtius rearrangement, an isocyanate, is trapped with benzyl alcohol to give the racemic tert-butyl 2-{[(benzyloxy)carbonyl]amino}-7-azabicyclo[2.2.1]heptane-7-carboxylate (7). This new compound was readily purified by crystallization, and the enantiomers thereof separated by chiral chromatography. A final hydrogenolysis affords 1 in high yield and high chemical and stereochemical purity.

Tropinone is converted into phenyl 3-oxobicyclo[3.2.1]octane-8-carboxylate (2) by dissolving tropinone is an inert organic reaction solvent, adding a weak insoluble inorganic base, conducting the reaction at a temperature of at least about 0° C., adding phenyl chloroformate, optionally heating to reflux after addition is complete, and isolating 2. 2 can be isolated by slowly adding an antisolvent, cooling the reaction from about −10° C. to about 20° C., collecting 2 by filtration, washing with a dilute acid solution, then washing with water, and optionally washing with a dilute base solution.

Step 1 is conducted in an inert organic solvent, including, but not limited to, toluene, acetonitrile, dichloromethane, or ethyl acetate; in the presence of a weak insoluble inorganic base, including, but not limited to, sodium or potassium bicarbonate or sodium or potassium carbonate; at 0° C. to 110° C. It is preferred to add phenyl chloroformate at a rate to keep the reaction temperature less than 30° C., and then after complete addition of the chloroformate, heat to reflux; it is preferred to heat in refluxing EtOAc. The methyl chloride that is a byproduct of the reaction is preferably scrubbed with a solution of aqueous KOH or NaOH in ethylene glycol, or with a solution of morpholine in aqueous ethanol.

Intermediate 2 is isolated by slowly adding an antisolvent, e.g., hexane or heptane, in an amount of about 1 to about 5 mL of antisolvent/mL of reaction solvent. It is preferred to have about 2 mL of antisolvent per mL of solvent. The reaction is then cooled from about −10° C. to about 20° C., preferably about 0° C., to crystallize 2. The solid is removed by filtration and washed with a dilute (from about 0.5% to about 5%) acid solution, including, but not limited to, sulfuric, phosphoric, or hydrochloric acid; hydrochloric acid is preferred. The solid is then washed with water, and may also be washed with a dilute (from about 0.5% to about 5%) base solution, including, but not limited to, bases such as potassium carbonate. Intermediate 2 can be obtained in a yield of about 85% with about 99% purity.

Phenyl 2-bromo-3-oxo-8-azabicyclo[3.2.1]octane-8-carboxylate (3) is obtained by dissolving 2 in an inert organic solvent, heating the reaction, adding anhydrous CuBr₂, and isolating 3. The halogenation of 2 is carried out in an inert organic solvent, including, but not limited to, toluene, acetonitrile, chloroform, an ethyl acetate/chloroform mixture or ethyl acetate at a concentration from about 5 mL to about 20 mL of solvent per gram of 2 (about 10 mL of solvent per gram of 2 is preferred and ethyl acetate is preferred), at a temperature from about 50° C. to about 110° C., it is preferred to conduct the reaction in refluxing in EtOAc. From about 1.9 to about 2.1 equivalents of anhydrous CuBr₂ is used; 2.0 equivalents are preferred. The reaction is allowed to stir from about 1 hour to about 24 hours, or until 2 is less than 1% by established analytical methods.

Intermediate 3 is isolated as a solution in toluene or another suitable solvent (from about 25 to about 75% w/v, preferably about 50% w/v) by filtering off the CuBr, washing the product solution with water and 5% aqueous NaHCO₃, removing the EtOAc in vacuo, and adding the suitable solvent, e.g., toluene, to give the desired concentration.

The Favorskii rearrangement to give 4 comprises dissolving 3 in water or an organic solvent, optionally adding 3 as a ca. 50% w/v solution in a suitable solvent, adding a base, and isolating 4, optionally having stereochemical purity of exo versus endo ratio being about 100:1. 3 is dissolved in either water or an organic solvent including, but not limited to, toluene, acetonitrile, dimethoxyethane, diethyl ether, methanol, ethanol, isopropanol, or benzyl alcohol; benzyl alcohol is preferred. From about 1 to about 3 eq of base are added, where the base includes, but is not limited to, sodium or potassium hydroxide, or the sodium or potassium salts of the identified alcoholic reaction solvents. The preferred base is sodium benzyloxide, with 2.2 equivalents of this base being preferred. The reaction is conducted at a concentration from about 3 mL to about 10 mL of solvent per gram of 3 (about 4 mL of solvent per gram of 3 is preferred). The use of benzyl alcohol as the solvent results in a dramatic and surprising improvement in the stereoselectivity of the Favorskii reaction, rendering a separate isomerization step or chromatographic separation of diastereoisomers unnecessary. Downstream crystallizations are also simplified, although the benzyl alcohol is difficult to remove and remains in the crude material taken into the next step. The reaction for Step 3 is conducted from about −10° C. to about 50° C. The preferred reaction temperature is about 5° C. The substrate is added to the base solution as a solution in toluene or another suitable solvent (ca. 50% w/v) at a rate to maintain the desired reaction temperature. The reaction is allowed to stir from about 1 hour to about 24 hours, or until less than 1% of bromoketone 3 or the corresponding benzyl carbamate remains by established analytical methods. The preferred reaction time is about 1 hour after the bromide addition is complete.

Intermediate 4 is isolated after the following workup procedure. The reaction mixture is diluted with 1:1 heptane/toluene and neutralized with conc. HCl. The product goes into the upper, organic phase. Residual phenol produced by the carbamate exchange is then washed away by extracting the organic phase with aqueous NaOH. After a brine wash, the product solution is dried (e.g., with Na₂SO₄), filtered and evaporated under reduced pressure at low temperature (e.g., about 40° C.). Most of the benzyl alcohol is then removed by distillation at about 5 mmHg with a bath temperature of up to 90° C. Intermediate 4 can be obtained with the exo versus endo ratio being about 100:1.

There are two different routes through which intermediate 6 can be obtained-5a or 5b. To obtain via 5a, 4 is dissolved in a low molecular weight alcohol or in a water-miscible solvent, treated with a base, and the reaction to stir from at least room temperature until 5a forms. 5a is isolated after a standard acid-base workup, dissolved with (BOC)₂O in a low molecular weight alcohol or other inert solvents, treated with a Pd/C catalyst under hydrogen gas from at least about atmospheric pressure, where additional (BOC)₂O is added where needed, and 6 is isolated.

The low molecular weight alcohol used to dissolve 4 includes, but not limited to, methanol, ethanol or isopropanol, and the water-miscible solvent includes, but not limited to, tetrahydrofuran or dioxane, in the presence of water. The reaction is conducted with about 3 to about 10 mL of solvent per gram of substrate (4), with the preference being about 4 mL of solvent per gram of 4 and ethanol being the preferred solvent.

Intermediate 4 is treated with about 1 to about 3 equivalents of base, preferably 1.5 eq., where the base includes, but is not limited to, sodium, potassium or lithium hydroxide, where sodium hydroxide is preferred. The reaction is conducted from about room temperature to about 80° C., with about 60° C. being preferred. The reaction is allowed to stir from about 10 minutes to about 24 hours, or until residual 4 is less than 1% relative to product 5a by an established analytical method. Intermediate 5a is isolated after a standard acid-base workup.

Intermediate 6 is obtained from 5a as follows: 5a and di-t-butyl dicarbonate ((BOC)₂O) are dissolved in a low molecular weight alcohol including, but not limited to, methanol, ethanol or isopropanol, or other inert solvents such as tetrahydrofuran; the preferred solvent is ethanol. Enough solvent is added to give a concentration of about 3 mL to about 10 mL of solvent per gram of substrate (5a), preferably about 6 mL/g. Pd/C is used as the catalyst, using about 5-10% Pd/C, preferably about 5% and using from about 0.05 to about 0.5 g of Pd/C per gram of substrate, preferably about 0.1 g Pd/C per g substrate. Hydrogen gas is applied from about atmospheric pressure (ca. 15 psi) to about 60 psi, preferably about 50 psi of hydrogen gas is applied. From about 1 to about 2 equivalents of di-t-butyl dicarbonate ((BOC)₂O) relative to substrate are used; preferably about 1.3 equivalents of (BOC)₂O. The reaction is conducted at a temperature of about 10° C. to about 50° C., preferably about 25° C. The reaction is run for about 1 hour to about 72 hours, or until 5a is less than 1% relative to product 6 by an established analytical method. Intermediate 6 is isolated by filtration of catalyst and evaporation of solvent, followed by a standard acid-base work-up.

Intermediate 6 is also obtained via 5b: dissolving 4 in a low molecular weight alcohol, using Pd/C, applying hydrogen (optionally at least about 30 psi), conducting the hydrogenolysis (optionally at a temperature that is at least room temperature), isolating 5b, dissolving 5b in THF and KOH (optionally 10% aqueous) to give a homogeneous solution, optionally adding aqueous KOH more than once, adding (BOC)₂O, optionally adding (BOC)₂O more than once, and isolating 6.

The low molecular weight alcohol in which to dissolve 4 includes, but not limited to, ethanol or isopropanol. Pd/C is used as the catalyst, using about 5-10% Pd/C, preferably about 5% and using from about 0.05 to about 0.5 g of Pd/C per gram of substrate (4), preferably about 0.1 g Pd/C per g substrate. From about 30 to about 60 psi of hydrogen gas is applied, preferably about 50 psi. The length of the reaction time depends on the purity (amount of residual benzyl alcohol) present with substrate 4, and can be from about 4 or 5 hours to several days. The reaction is run from about room temperature to about 50° C. The catalyst is removed by filtering through Celite, followed by evaporation of the solvent. The resulting material is partitioned between water and ethyl acetate, with the amine going into the aqueous layer. To recover the product, a solvent switch from water to isopropanol is performed. The isopropanol-water azeotrope is distilled off until an anhydrous solution of the amino acid in isopropanol is obtained. Addition of ethyl acetate to this solution induces crystallization of the product, which is then collected by filtration, washed with ethyl acetate and dried. This procedure can give a very pure product as a free-flowing, nearly white solid. The removal of all of the water prior to crystallization is believed to be critical to obtaining a nice solid.

The amino acid 5b is converted into the BOC acid 6 by dissolving the amino acid in THF and about 10% aqueous KOH (at least 1 equiv) in proportions that gives a homogeneous solution, and adding from about 1 to about 2 equivalents of (BOC)₂O, preferably about 1.2 equivalents, either neat or as a THF solution. The mixture is stirred from about room temperature to about 40° C. until all of the amino acid is consumed, additional KOH and (BOC)₂O may be needed for the reaction to go to completion. The THF is distilled out since it would otherwise interfere in the subsequent phase separations. The residue is partitioned between ethyl acetate and water. The aqueous phase containing the potassium salt of the product is then acidified to pH 3 with 2 M HCl to precipitate the free acid, while the aqueous phase is kept at a temperature from about room temperature to about 5° C. The solid is collected by filtration, washed with water and dried to give a white solid of very high purity. Intermediate 6 is obtained in yields ranging from about 67% to about 75%.

Step 6 is a Curtius rearrangement. 6 and a non-nucleophilic organic soluble base are dissolved in a high-boiling, inert organic solvent, the solution is heated, DPPA is added preferably at a rate to control the reaction temperature and nitrogen off-gassing rate, the reaction is heated from about 30° C. to about 110° C. once the temperature and nitrogen off gassing begins to fall, benzyl alcohol is added, the reaction at from about 30° C. to 110° C., until residual 6 is less than 1% relative to (racemic) exo-tert-butyl 2-[(benzyloxy)carbonyl]amino-7-azabicyclo[2.2.1]heptane-7-carboxylate (7), further comprising a mildly basic aqueous work-up, further comprising evaporation of the solvent, and further comprising filtering the reaction through a pad of silica gel and eluting with 40-50% ethyl acetate in hexane.

More specifically, 6 and a non-nucleophilic, organic soluble base (from about 1 to about 1.5 equivalents relative to substrate (6)), including, but not limited to, triethylamine or diisopropyl ethylamine (preferably 1.05 eq of triethyl amine are used), are dissolved in a high-boiling, inert organic solvent, including, but not limited to, toluene or xylene to give a resulting concentration of solvent to substrate of about 3 mL to about 10 mL of solvent per gram of substrate, preferably about 7 mL of solvent per gram of substrate. The solution is heated from about 30° C. to about 80° C. and DPPA (from about 0.95 to about 1.2 equivalents, preferably about 1.05 equivalents) is slowly added at a rate to control the reaction temperature and nitrogen off-gassing rate. When the temperature and off-gassing rate start to drop, the mixture is heated from about 30° C. to about 110° C., preferably about 80° C., treated with benzyl alcohol (from about 0.95 to about 1.5 eq, preferably about 1.0 eq), and stirred at a temperature from about 30° C. to about 110° C., preferably about 80° C., for about 1 hour to about 24 hours, or until residual 6 is less than 1% relative to tert-butyl 2-{[(benzyloxy)carbonyl]amino}-7-azabicyclo[2.2.1]heptane-7-carboxylate (7) by an established analytical method. After a mildly basic (e.g., NaHCO₃) aqueous work-up, the solvent is evaporated and the product filtered through a pad of silica gel, eluting with 40-50% ethyl acetate in hexane. If too much benzyl alcohol remains, it is removed azeotropically by distillation with mesitylene. The product is then crystallized from hexane/ethyl acetate.

Step 7 is the chiral separation of each enantiomer of intermediate 7: In order to simplify the chiral separation, by-products uronyl azide (10), and the phosphorus compound 11 and reagent benzyl alcohol are first separated out by reversed phase HPLC. Alternatively, the chiral separation may be simplified by first crystallizing the racemic 7 from hexane/EtOAc. The chiral separation then proves to be straightforward. Each enantiomer is obtained in high purity and recovery off of a Chiralcel OD column (e.g., from Daicel) eluted with heptane/iPrOH/TFA. The chiral purity of 8 is estimated to be about >99.5%, with chemical purity also being very high.

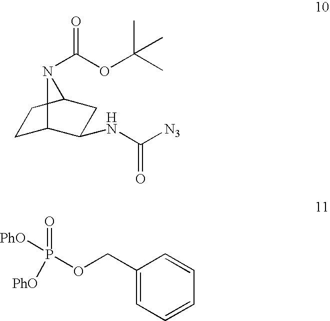

Another aspect of the present invention is the separation of the enantiomers of 7 using continuous chromatography, semi-continuous chromatography or single column chromatography. Some of the examples of semi-continuous chromatography are liquid chromatography technologies known by the names cyclojet, SteadyCycle or Steady State Recycling (U.S. Pat. Nos. 6,063,284 and 5,630,943). Examples of continuous chromatography are liquid chromatography techniques known as simulated moving bed chromatography (SMB). The concept of SMB has been described in U.S. Pat. Nos. 2,957,927 and 2,985,589 and has long been used in the petrochemical and sugar industries. See, Nicoud, R. M., LC-GC Intl, 5 (5) 43 (1992).

SMB combines the high-resolution power of high performance liquid chromatography (HPLC) with the lower cost of classical separation processes such as crystallization and distillation. The costs of the SMB process can be reduced even further if combined with a racemization step that converts the inactive enantiomer into the racemic form, which can then be recycled back into the SMB process. The cost of the SMB process can also be reduced by coupling the SMB separation with a crystallization to increase the optical purity.

The chromatography comprises a liquid mobile phase and a solid chiral stationary phase. The solid stationary phase is selected from the following: 1) amylosic, cellulosic, xylan, curdlan, dextran or inulan class of polysaccharides, 2) amylosic, cellulosic, xylan, curdlan, dextran or inulan class of polysaccharides coated or adsorbed on silica gel, zirconium, alumina, ceramics and other silicas, 3) amylosic, cellulosic, xylan, curdlan, dextran or inulan class of polysaccharides chemically bound to silica gel, zirconium, alumina, ceramics and other silicas, 4) derivatized silica sorbents (Pirkle type), 5) tartaric acid derivatives or 6) other stationary phases containing chiral molecules. The mobile phase contains C₁₋₅ alcohols and C₁₋₁₀ hydrocarbons. Also acetonitrile, methyl acetate, ethyl acetate, methylene chloride, toluene, methyl tert-butyl ether and/or mixtures thereof. In addition, the mobile phase can be subcritical or supercritical CO₂ in combination with C₁₋₁₀ alcohols, acetonitrile, ethyl acetate, methyl acetate, methylene chloride, toluene, methyl tert-butyl ether and/or mixtures thereof. The temperature range is from about 5 to about 45° C., preferable about 20 to 40° C.

1 is then obtained from 8: 8 is dissolved in a low molecular weight alcohol or inert solvent; Pd/C is added, optionally 5-10% and further optionally adding from about 0.05 to about 0.5 g Pd/C per gram of 8; hydrogen gas is applied, optionally from about atmospheric pressure (ca. 15 psi) to about 60 psi; the reaction is allowed to proceed, preferably until 8 has been consumed; and 1 is isolated, optionally having at least 95% chemical purity and at least about 95% chiral (enantiomeric) purity.

The low molecular weight alcohol in which 8 is dissolved, includes, but is not limited to, methanol, ethanol or isopropanol, or in some other inert solvent, for example, but not limitation, tetrahydrofuran; the preferred solvent is ethanol. Solvent is used to give from about 3 mL to about 10 mL of solvent per gram of substrate (8), preferably 6 mL/g. Pd/C is used, preferably 5-10%, more preferred 5% Pd/C, using from about 0.05 to about 0.5 g Pd/C per gram of substrate, preferably about 0.1 g Pd/C per gram of substrate. Hydrogen gas is applied from about atmospheric pressure (ca. 15 psi) to about 60 psi, preferably 50 psi, and the mixture is agitated, e.g., until the starting material 8 has been consumed. The product is isolated by filtration (to remove the catalyst) followed by evaporation of the solvent. Further purification is not necessary. Compound 1 is obtained optionally having at least 95% chemical purity and at least about 95% enantiomeric purity. It is preferred that 1 has at least about 97% chemical purity and at least about 99.5% chiral (enantiomeric) purity.

Another aspect of this invention includes a compound that is dibenzyl 7-azabicyclo[2.2.1]heptane-2,7-dicarboxylate (4) of varying degrees of chemical purity and chiral purity. Another aspect of this invention includes a compound that is exo-(t-butyl-2(R(+))-amino-7-azabicyclo[2.2.1]heptane-7-carboxylate (1); exo-tert-butyl 2(R(+)-{[(benzyloxy)carbonyl]amino}-7-azabicyclo[2.2.1]heptane-7-carboxylate (8); exo-tert-butyl 2-[(benzyloxy)carbonyl]amino-7-azabicyclo[2.2.1]heptane-7-carboxylate (7); (2R)-7-(tert-butoxycarbonyl)-7-azabicyclo[2.2.1]heptane-2-carboxylic acid (15); or 7-(tert-butoxycarbonyl)-7-azabicyclo[2.2.1]heptane-2-carboxylic acid (6), each of which has varying degrees of chemical purity and chiral purity, optionally having chiral purity of at least 90%, e.g., 90% or higher, for example, 98% or 99%, and further optionally having chemical purity of at least 95% e.g., 95% or higher, for example, 99% or higher, like 99.5%. Furthermore, the compounds can be alkali metal or amine (chiral or achiral) salts of the acid, or acid (chiral or achiral) salts of the amine.

Another aspect of the present invention includes the enantiomeric resolution of 7-aza-bicyclo[2.2.1]heptane 2,7-dicarboxylic acid dibenzyl ester (4) to give 4 in varying degrees of chemical purity and chiral purity. For example, but not limitation, this is done by dissolving about 20 grams of 60% pure 7-aza-bicyclo[2.2.1]heptane 2,7-dicarboxylic acid dibenzyl ester in about 25 mL DMSO in a reaction vessel, adding about 500 mL 1 M sodium phosphate (pH 7.1), and adding about 240 grams Amano AY and stirring at about room temperature using 2.5 cm marine prop, 335 rpm. After about nine days, the agitation is ceased and aqueous layer decanted from the precipitate. The aqueous layer is centrifuged, and the pellet from the centrifuge is combined with the precipitate in the reactor. The combined pellet and precipitate is extracted with first ethyl acetate and then with ethanol. The extracts are combined and dried thoroughly to give 5.25 of the exo isomer of 4 ((+) stereoisomer. By assay, the enantiomer is about 64% pure by area percent. A chiral assay indicates that it has 94% (89% ee) chiral purity.

Another aspect of the present invention includes the enantiomeric separation of 7-aza-bicyclo[2.2.1]heptane 2,7-dicarboxylic acid dibenzyl ester using chiral column chromatography. For example, but not limitation, the exo desired enantiomer compound can be isolated using a Chiralpak AD using a mobile phase of 100/0.1 ethanol/trifluoracetic acid.

Another aspect of the present invention includes the classical resolution of the enantiomers of 7-[(benzyloxy)carbonyl]-7-azabicyclo[2.2.1]heptane-2-carboxylic acid (5a) of varying degrees of chemical purity and chiral purity, optionally having chemical purity being at least 90% and chiral purity being at least 90% or higher, e.g., 95% or even 99%. For example, crude 5a (10.0 g, 36.4 mmol) is combined with 60 mL of EtOAc in a round bottom flask. The mixture is heated to about 65° C. to dissolve the solid. R(+)-a-methylbenzylamine (2.2 g, 18.2 mmol) is added to the hot mixture via syringe over ca. 1 min. The resulting solution is stirred at about 65° C. for about 10 min, then allowed to cool slowly to room temperature and stir overnight. The resulting solid is isolated by suction filtration and dried in a vacuum oven to afford 3.2 g of white crystals (22.2% recovery based on 50% maximum yield). The solid assayed as a 96.4/3.9 ratio of diastereoisomers by chiral HPLC on a Chiralcel OD column eluted with 95/5 heptane/isopropanol containing 0.1% TFA. The salt (0.50 g) is recrystallized by dissolving it in EtOAc (8 mL), diluting the solution with 3 mL of hexane, heating to about 65° C., and allowing the mixture to cool slowly to room temperature and stir overnight. The resulting mixture is suction filtered and the cake dried in a vacuum oven to furnish 0.40 g of white crystals (80% recovery). The recrystallized material is determined to be a 114/1 ratio of diastereomers when assayed as above. The resolved, free carboxylic acid could be isolated in quantitative yield by partitioning between EtOAc and dilute (e.g., from about 1% to about 10%) aqueous HCl. The free acid is recovered from the EtOAc layer.

Another aspect of the present invention includes the final compounds, the intermediates, and the resolution of the final compound or intermediates using the methods discussed herein such that the compounds are of varying degrees of stereochemical purity.

Another aspect of the present invention includes the enantiomeric separation of 7-(tert-butoxycarbonyl)-7-azabicyclo[2.2.1]heptane-2-carboxylic acid (6) using a Chiralpak AS or Chiracel OJ column using a mobile phase of 5/95/0.1 of IPA/heptane/TFA.

Another aspect of the present invention includes the enantiomeric separation of any intermediate after intermediate 3 discussed in Scheme 1 and herein. The sooner the separation occurs, the more efficient the preparation of 1. Only one enantiomeric separation is needed.

Not only is the route in Scheme 1 surprisingly improved, but other routes have also been identified. A second strategy involves using a t-butyl carbamate (BOC) group early. However, because the BOC group is not stable to the bromination conditions, a new halogenation method had to be developed. This involves formation of a silyl enol ether, and the chlorination thereof. This procedure also circumvents the scalability problems associated with the bromination procedure.

Another aspect of the present invention includes the preparation of 1 according to the process as outlined in Scheme 2, including, but not limited to, any single step or more than one step within Scheme 2 in combination with any other single step discussed herein. The desired compound 1 can be obtained according to Scheme 2:

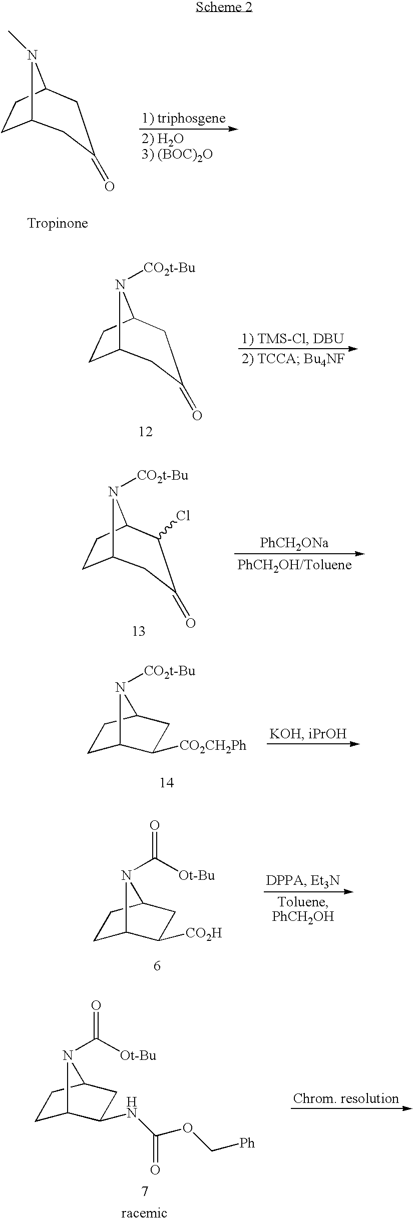

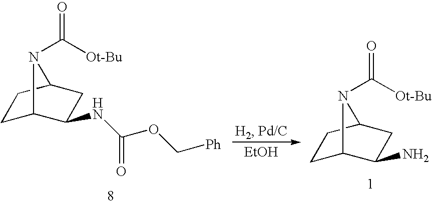

In this route, the BOC group is used at the beginning by modifying procedures discussed in GB 1,167,688. Since the resulting tert-butyl 3-oxo-8-azabicyclo[3.2.1]octane-8-carboxylate (12) would not be stable to the CuBr₂-mediated bromination conditions (which produce hot HBr), an alternate halogenation procedure was developed. This involves conversion of ketone 12 into the corresponding silyl enol ether with trimethylsilyl chloride and an amine base (diazabicycloundecene, DBU), followed by treatment of the silyl enol ether intermediate with a chlorinating agent (trichlorotriazinetrione, TCCA). Finally, residual silyl groups are removed by treatment of the mixture with Bu₄NF to give tert-butyl 2-chloro-3-oxo-8-azabicyclo[3.2.1]octane-8-carboxylate (13). Submission of chloroketone 13 to the previously mentioned Favorskii rearrangement conditions again furnishes the rearranged 2-benzyl 7-tert-butyl 7-azabicyclo[2.2.1]heptane-2,7-dicarboxylate (14) in good yield and with very high stereoselectivity in favor of the exo isomer. Hydrolysis of the benzyl ester gives intermediate 6, which is converted into 1 in the same manner as described in Scheme 1 above.

Another aspect of the present invention includes a process for preparing 1 from tropinone, comprising any one single step or combination of sequential steps of the following:

further comprising preparing tert-butyl 3-oxo-8-azabicyclo[3.2.1]octane-8-carboxylate (12) from tropinone using triphosgene, followed by the addition of water, neutralization with aqueous NaOH, and then addition of (BOC)₂O;

further comprising preparing tert-butyl 2-chloro-3-oxo-8-azabicyclo[3.2.1]octane-8-carboxylate (13) from 12 using TMS-Cl, DBU followed by TCCA and Bu₄NF;

further comprising preparing 2-benzyl 7-tert-butyl 7-azabicyclo[2.2.1]heptane-2,7-dicarboxylate (14) from 13 using PhCH₂ONa in PhCH₂OH/toluene;

further comprising preparing 7-(tert-butoxycarbonyl)-7-azabicyclo[2.2.1]heptane-2-carboxylic acid (6) from 14 using KOH in isopropanol;

further comprising preparing tert-butyl 2-{[(benzyloxy)carbonyl]amino}-7-azabicyclo[2.2.1]heptane-7-carboxylate (7) from 6 using DPPA and Et₃N in toluene and PhCH₂OH;

further comprising resolving exo-tert-butyl 2(R(+)-{[(benzyloxy)carbonyl]amino}-7-azabicyclo[2.2.1]heptane-7-carboxylate (8) from 7, for example, using methods discussed herein; and

further comprising preparing 1 from 8 by hydrogenolysis in the presence of Pd/C in an alcoholic solvent including ethanol. For example, and not limitation, the invention may include every step listed or may be less than all of the steps listed.

Another aspect of the present invention includes the preparation of 1 according to the process as outlined in Scheme 3, including, but not limited to, any single step or more than one step within Scheme 3, individually, or in combination with any other step discussed herein. The desired compound 1 can be obtained according to Scheme 3:

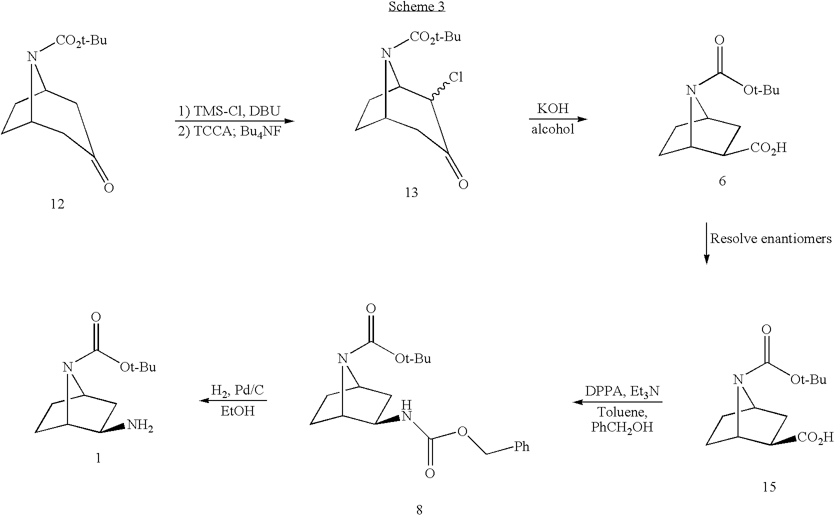

In this route, protected amino ketone 13, prepared from 12, see, e.g., Scheme 2, is treated with aqueous KOH in an alcoholic solvent such as isopropanol or ethanol to induce the Favorskii rearrangement. Under these new conditions, the carboxylic acid 6 is produced directly, perhaps at least partly through an intermediate isopropyl ester. Alternatively, and preferably, 6 can be obtained from 12 from the chlorinated silyl enol ether intermediate (immediate precursor to 13) by not using the tetrabutylammonium fluoride and without having to purify the enol intermediate other than isolating it in the organic phase using standard work-up procedures. This unexpected finding (hydroxide is normally a poor base for the Favorskii rearrangement) eliminates a step from the synthesis. Compound 6 may then be converted into 1 by the sequences described earlier. Alternatively, and preferably, 6 may be resolved into its separate enantiomers prior to conversion into 1. This may be accomplished chromatographically, as described herein. Alternatively, the enantiomers may be resolved classically by forming diastereomeric salts of 6 with stereochemically pure amines such as α-methylbenzylamine. Selective crystallization of the desired diastereomer, followed by liberation of the desired enantiomer of 6 by treatment with acid completes the resolution. Finally, racemic 6 may be treated with an alcohol in the presence of an enzyme such as a lipase. This promotes esterification of the undesired enantiomer of 6, leaving the desired enantiomer to be separated by simple acid-base extractions. These same enantiomer resolution strategies apply to the other routes described above as well. In addition, the esters 4 and 14 (Schemes 1 and 2, respectively) produced by the Favorskii rearrangement, may be resolved by enantioselective enzymatic hydrolysis of the desired enantiomer of these esters.

Another aspect of the present invention includes a process for preparing 6 from tropinone, comprising any one single step or combination of sequential steps of the following or with any other step(s) discussed herein:

Preparing 7-(tert-butoxycarbonyl)-7-azabicyclo[2.2.1]heptane-2-carboxylic acid (6) from tropinone, comprising preparing tert-butyl 3-oxo-8-azabicyclo[3.2.1]octane-8-carboxylate (12) from tropinone, comprising addition of triphosgene to tropinone in an inert solvent, optionally toluene;

further comprising addition of water;

further comprising adding an aqueous base, optionally NaOH, to raise the reaction pH, optionally above 10;

further optionally comprising the addition of more toluene; and

further comprising addition of (BOC)₂O, optionally comprising the addition of DMAP to catalyze the destruction of residual (BOC)₂O and isolation of 12;

further comprising treating 12 with an amine base, wherein the base is optionally diazabicycloundecene or DBU (DBU is preferred), and TMS-Cl to give a silyl enol ether intermediate;

further comprising treating the enol intermediate in EtOAc with TCCA to give a chlorinated intermediate;

further optionally comprising isolating the chlorinated intermediate and optionally removing EtOAc and toluene;

further comprising treating the chlorinated intermediate with a base including KOH in an alcoholic solvent, optionally isopropanol, or NaOH in an alcoholic solvent, optionally ethanol, NaOH is preferred.

Another aspect of the present invention includes a process for preparing 1 from 6, comprising any one single step or combination of sequential steps of the following or with any other step(s) discussed herein:

Preparing exo-tert-butyl 2(R(+)-{[(benzyloxy)carbonyl]amino}-7-azabicyclo[2.2.1]heptane-7-carboxylate (8) from 6, comprising either resolving (2R)-7-(tert-butoxycarbonyl)-7-azabicyclo[2.2.1]heptane-2-carboxylic acid (15) from 6 using the methods discussed herein, or resolving 8 from tert-butyl 2-{[(benzyloxy)carbonyl]amino}-7-azabicyclo[2.2.1]heptane-7-carboxylate (7) by preparing 7 from 6;

further comprising preparing 8 from 15 or preparing 7 from 6 comprising treating either 15 or 6 with DPPA and a non-nucleophilic organic soluble base, optionally Et₃N, in a high-boiling, inert organic solvent, optionally toluene and PhCH₂OH; and

further comprising preparing 1 from 8 by hydrogenolysis in the present of Pd/C in an alcoholic solvent including ethanol.

Another aspect of the present invention includes a process for preparing 1 from tropinone, comprising any one single step or combination of sequential steps of the following or with any other step(s) discussed herein:

Preparing 6 from tropinone, comprising treating tert-butyl 3-oxo-8-azabicyclo[3.2.1]octane-8-carboxylate first with neat DBU, optionally 1.4 eq, and then followed by the addition of neat TMS-Cl, optionally 1.3 eq, relative to the carboxylate;

further comprising isolating the enol intermediate, including isolating the crude enol intermediate in an organic phase and treating it without further purification;

further comprising the addition of solid TCCA to the enol intermediate in EtOAc optionally cooled to about 0-5° C., and further optionally stirring at 0° C. until the enol intermediate is consumed; and

further comprising isolating the chlorinated intermediate and removing EtOAc and toluene.

Another aspect of the present invention includes a process for preparing 1 from tropinone, comprising any one single step or combination of sequential steps of the following or with any other step(s) discussed herein:

Preparing exo-tert-butyl 2(R(+)-{[(benzyloxy)carbonyl]amino}-7-azabicyclo[2.2.1]heptane-7-carboxylate (8) from 6, comprising either resolving (2R)-7-(tert-butoxycarbonyl)-7-azabicyclo[2.2.1]heptane-2-carboxylic acid (15) from 6, or resolving 8 from preparing tert-butyl 2-{[(benzyloxy)carbonyl]amino}-7-azabicyclo[2.2.1]heptane-7-carboxylate (7) after preparing 7 from 6;

further comprising preparing 8 from 15 or preparing 7 from 6 comprising treating either 15 or 6 with DPPA and a non-nucleophilic, organic soluble base, optionally Et₃N, in a high-boiling, inert organic solvent, optionally toluene, and PhCH₂OH; and

further comprising preparing 1 from 8 by hydrogenolysis in the present of Pd/C in an alcoholic solvent including ethanol.

Another aspect of the present invention includes a process for preparing 8 from 6, comprising any one single step or combination of sequential steps of the following or with any other step(s) discussed herein:

Comprising resolving 15 from 6;

further comprising dissolving 6 or 15 and from about 1 eq to about 1.5 eq of Et₃N, in toluene;

further comprising heating the solution, optionally from about 30° C. to about 80° C.;

further comprising adding DPPA, optionally from about 0.95 eq to about 1.2 eq, and further optionally at a rate to control the reaction temperature and nitrogen off-gassing rate;

further comprising heating the reaction, optionally from about 30° C. to 110° C. once the temperature and nitrogen off gassing begins to fall;

further comprising adding benzyl alcohol, optionally from about 0.95 eq to about 1.5 eq;

further comprising stirring the reaction, optionally at a temperature from about 30° C. to 110° C., until residual 6 or 15 is less than 1% relative to 7 or 8, respectively;

further comprising a mildly basic aqueous work-up, optionally purifying using silica gel chromatography eluting with EtOAc, optionally 40-50% EtOAc in hexane; and

further comprising isolating 7 or 8, and resolving 8 from 7.

Further aspects and embodiments of the invention may become apparent to those skilled in the art from a review of the following detailed description, taken in conjunction with the examples and the appended claims. While the invention is susceptible of embodiments in various forms, described hereafter are specific embodiments of the invention with the understanding that the present disclosure is intended as illustrative, and is not intended to limit the invention to the specific embodiments described herein.

### DETAILED DESCRIPTION

The present invention provides a safer, scalable, and more efficient route relative to current routes for the preparation of a key intermediate having absolute 1S, 2R, 4R or exo-2R stereochemistry on the azabicyclic ring system of the 2-amino-7-azabicyclo[2.2.1]heptane intermediate 1. One aspect of the present invention includes the synthesis of the 2-amino-7-azabicyclo[2.2.1]heptane intermediate 1 as discussed herein.

The key and novel features of this route are the use of a phenyl carbamate protecting group for the amino group of tropinone, and careful selection of the reaction conditions for the subsequent Favorskii ring-contraction step. The phenyl carbamate group proved to be readily introduced and stable to the bromination conditions used in the process. In addition, this group is efficiently converted into a benzyl carbamate group during the Favorskii step (an uncommon example of an intermolecular carbamate exchange reaction), which could be removed later by catalytic hydrogenation. The critical Favorskii rearrangement step is carried out with sodium benzyloxide as the base in benzyl alcohol as the solvent. These conditions not only induced the desired carbamate exchange, but also unexpectedly furnished the rearranged product in a highly stereoselective manner. Together, these findings uniquely allowed a safe, efficient, and scalable process to be developed.

Not only is this route surprisingly improved, but other routes are also identified. A second strategy involves using a t-butyl carbamate (BOC) group early. However, because the BOC group is not stable to the bromination conditions, a new halogenation method had to be developed. This involves formation of a silyl enol ether, and the chlorination thereof. This procedure also circumvents the scalability problems associated with the bromination procedure.

Finally, a third route (Scheme 3) was developed that incorporates the early t-butyl carbamate (BOC) group introduction and the new halogenation procedure of Scheme 2, and also employs improved conditions for the Favorskii rearrangement. The Favorskii rearrangement of the chlorinated silyl enol ether derived from 12 was unexpectedly found to be efficiently promoted by aqueous, alcoholic sodium or potassium hydroxide. The advantages of these new conditions are that the difficult to remove benzyl alcohol is no longer needed, and the product is a more advanced intermediate, carboxylic acid 6. Thus, a separate hydrolysis step is avoided. In addition, with the new procedure carboxylic acid 6 is obtained from tropinone without isolation of any intermediates, and the Favorskii rearrangement proceeds with equally high stereoselectivity in favor of the desired exo isomer.

Abbreviations which are well known to one of ordinary skill in the art may be used (e.g., “Ph” for phenyl, “Me” for methyl, “Et” for ethyl, “h” or “hr” for hour or hours, min for minute or minutes, and “rt” or “RT” for room temperature).

All temperatures are in degrees Centigrade.

Room temperature is within the range of 15-25 degrees Celsius.

FLIPR refers to a device marketed by Molecular Devices, Inc. designed to precisely measure cellular fluorescence in a high throughput whole-cell assay. (Schroeder et. al., J. Biomolecular Screening, 1(2), p 75-80, 1996).

1 and Formula 1 are used interchangeably and both refer to exo-(t-butyl 2R(+))-2-amino-7-azabicyclo[2.2.1]heptane-7-carboxylate.

TLC refers to thin-layer chromatography.

HPLC refers to high pressure liquid chromatography.

MeOH refers to methanol.

EtOH refers to ethanol.

IPA refers to isopropyl alcohol.

THF refers to tetrahydrofuran.

DMSO refers to dimethylsulfoxide.

DMF refers to N,N-dimethylformamide.

EtOAc refers to ethyl acetate.

TMS refers to tetramethylsilane.

TEA refers to triethylamine.

DIEA refers to N,N-diisopropylethylamine.

MLA refers to methyllycaconitine.

Ether refers to diethyl ether.

CDI refers to carbonyl diimidazole.

NMO refers to N-methylmorpholine-N-oxide.

TPAP refers to tetrapropylammonium perruthenate.

Halogen is F, Cl, Br, or I.

Na₂SO₄ refers to sodium sulfate.

K₂CO₃ refers to potassium carbonate.

MgSO₄ refers to magnesium sulfate.

When Na₂SO₄, K₂CO₃, or MgSO₄ is used as a drying agent, it is anhydrous.

DBU refers to 1,8-diazabicyclo[5.4.0]undec-7-ene.

TMS-Cl refers to trimethylchlorosilane.

TCCA refers to trichloroisocyanuric acid.

DMAP refers to 4-dimethylaminopyridine.

DPPA refers to diphenylphosphoryl azide.

The carbon atom content of various hydrocarbon-containing moieties is indicated by a prefix designating the minimum and maximum number of carbon atoms in the moiety, i.e., the prefix Cᵢ₋ⱼ indicates a moiety of the integer “i” to the integer “j” carbon atoms, inclusive. Thus, for example, C₁₋₆ alkyl refers to alkyl of one to six carbon atoms.

Mammal denotes human and other mammals.

Brine refers to an aqueous saturated sodium chloride solution.

Equ means molar equivalents.

IR refers to infrared spectroscopy.

Lv refers to leaving groups within a molecule, including Cl, OH, or mixed anhydride.

NMR refers to nuclear (proton) magnetic resonance spectroscopy, chemical shifts are reported in ppm (δ) downfield from TMS.

MS refers to mass spectrometry expressed as m/e or mass/charge unit. HRMS refers to high resolution mass spectrometry expressed as m/e or mass/charge unit. [M+H]⁺ refers to an ion composed of the parent plus a proton. [M−H]⁻ refers to an ion composed of the parent minus a proton. [M+Na]⁺ refers to an ion composed of the parent plus a sodium ion. [M+K]⁺ refers to an ion composed of the parent plus a potassium ion. EI refers to electron impact. ESI refers to electrospray ionization. CI refers to chemical ionization. FAB refers to fast atom bombardment.

GC-MS refers to gas chromatograph-mass spectrometry. A Hewlitt-Packard instrument comprised of a model 5890 gas chromatograph coupled to a model 5970 mass spectrometer was used. The column was a 30 m DB-5 column from Alltech, operated from 100-290° C.

Another aspect of the present invention includes the final compounds, the intermediates, and the resolution of the final compound or intermediates using the methods discussed herein such that the compounds are of varying degrees of stereochemical purity.

### Preparation of 1

8-(Phenoxycarbonyl)-8-azabicyclo[3.2.1]octane-3-one:

Solid tropinone (75 g, 0.54 mol) is dissolved in EtOAc (225 mL) in a 1 L jacketed reactor and treated with solid K₂CO₃ (0.75 g). The reactor is fitted with a mechanical stirrer, a nitrogen inlet, a thermocouple, and a water-cooled reflux condenser. A line from the top of the condenser leads the nitrogen/CH₃Cl stream to a scrubber containing morpholine, ethanol and water. With the jacket at 20° C. and the reaction mixture at 17° C., the addition of neat phenyl chloroformate (68 mL, 0.54 mol) through a dropping funnel is started. The chloroformate is added over 15 min, so that the reaction temperature remains below 25° C. When the mild exotherm subsides, the mixture is slowly warmed to 50° C., and held at 50° C. for about 1 h, or until essentially all of the tropinone and intermediates are consumed (TLC, ca. 2:1 hexane/EtOAc). The mixture is diluted with heptane (450 mL, added slowly). The jacket is then set to 25° C., and the mixture is cooled slowly to rt. The mixture is then cooled to 0° C. over 1.25 h, held there for 10 min, and then treated with 50 mL of H₂O. The entire mixture is suction filtered on a Büchner funnel (Whatman #1 filter paper), and the filter cake is washed with 2:1 heptane/EtOAc and sucked dry for a few minutes. The vacuum is disconnected, and the cake is washed once with 0.5% HCl and once with water, allowing ca. 5 min contact each time before sucking the water through.

The resulting solid is air dried for 30 min, then placed in a vacuum oven at 50° C. to constant weight. The yield of 2 (PNU-144240) is 110 g (83% yield). The material is 99.4% purity by GC area %, the major impurity being tropinone (0.4%).

Bromination of Nortropinone Phenyl Carbamate:

A 20 L jacketed reactor is equipped with an overhead stirrer, a thermocouple, a nitrogen inlet in one neck, and a water-cooled condenser. Outlet lines are connected to another neck and to the top of the condenser. Each outlet line leads through an empty trap (to prevent back-up), then to a mineral oil bubbler, and finally to a gas sparger immersed in ˜8% NaOH solution. Substrate 2 (750 g, 3.054 mol) and CuBr₂ (1.362 kg, 6.107 mol) are charged to the reactor, each being rinsed in with EtOAc (250 mL). The remaining EtOAc (7 L) is added, and the mixture is stirred at 170 rpm while the temperature was raised to ca. 75° C. over about 1 h. At this point, a color change and the appearance of HBr vapor in the scrubber head space indicates that the reaction has started. The mixture is stirred for an additional 1 h at ca. 75° C., whereupon the dark solid CuBr₂ is consumed and replaced by a brown solid (CuBr). A sample is taken, and this shows a ratio of starting material/product/dibromide of 10.8/85.6/3.6 by GC area %. A second sample taken 30 min later shows 9.2/85.3/4.7. The reaction mixture is cooled to rt over 4 h, and allowed to stir at rt overnight. The reaction mixture is suctioned out of the reactor into 4 L filter flasks. The residue in the reactor is washed twice with toluene, each wash being also suctioned out. The CuBr is removed by filtration through a sintered glass filter funnel, and rinsed thoroughly with toluene. The filtrate is transferred to a 35 L reactor for the work-up, rinsing it in with a little toluene (a total of 4 L of toluene was used). The crude reaction mixture is stirred vigorously with 3 kg of water for 20 min, then the phases are allowed to separate and settle for 10 min. The green aqueous phase (pH 1, 3.031 kg) is drained out, and the organic phase is washed similarly with 3 kg of 5% aqueous NaHCO₃. After draining this wash (pH 7, 3.194 kg), the organic phase is drained into 4 L Erlenmeyer flasks and dried over Na₂SO₄. The product solution is suction filtered through celite, and the cake rinsed with toluene. The solution is concentrated on a rotary evaporator at ≦40° C. under reduced pressure down to about 2 L total volume. GC analysis indicates that EtOAc is still present. The mixture is diluted with 1 L of toluene and then evaporated down to ˜1 L total volume as above, whereupon EtOAc is absent according to GC analysis. The dark brown product solution is then diluted to 2 kg total weight with toluene, and is ready for use in the next step. MS (GC-MS) m/z (rel. intensity) 325 (M⁺, 2), 323 (M⁺, 2), 244 (100), 232 (32), 230 (32), 188 (45), 110 (11), 94 (34), 79 (28).

Favorskii Rearrangement:

A 35 L jacketed reactor is equipped with an overhead stirrer, a thermocouple, a nitrogen inlet, and a dropping funnel. The reactor is charged with the NaOCH₂Ph (26.7% solution in benzyl alcohol, 6.21 kg, 12.76 mol) and benzyl alcohol (1.33 kg), and the viscous mixture is stirred at 150 rpm and cooled to ˜5° C. While the mixture is cooling, the toluene solution of crude bromide 3 (˜1.5 kg, 5.102 mol contained) is charged to the dropping funnel. The bromide solution is added to the reaction mixture over 50 min, with a maximum reaction temperature of 8° C. The dropping funnel is rinsed with a little toluene, and this is also added to the reaction mixture. After stirring the reaction mixture for 15 min at about 0° C., a sample is taken. This showed complete conversion to 4 (GC area %). Toluene (4 L) and heptane (4 L) are then added sequentially, and the dropping funnel is charged with a solution comprised of 750 g of conc. HCl diluted with 3.25 kg of water. The aqueous acid is added in dropwise over about 20 min, the last ˜1 L being added very rapidly since the exotherm subsides. The maximum temperature during the quench is 13° C. The resulting mixture is stirred vigorously for 20 min, then the agitator is stopped and the phases are allowed to separate and settle for about 1 h. The aqueous phase is drained, and the organic phase is washed with 6 kg of 5% KOH. Cooling is applied to keep the temperature at about 15° C. during the wash. The mixture is stirred vigorously for about 20 min, then the agitator is turned off and the phases are allowed to separate and settle. The separation is very slow—about 2.5 h. The aqueous phase is drained (6.591 kg), and the organic layer washed again with 3.275 kg of 2.5% KOH. This time, the aqueous phase (3.627 kg) may be drained after only 15 min settling time. Finally, the organic phase is washed sequentially with water (4 kg) and brine (3.22 kg), both phase separations being clean and rapid. The product solution is drained into a tared 5-gallon drum. The solution weighs 15.736 kg and is assayed as 5.95% product by weight. This corresponds to 936 g of contained product, or 50% overall yield from 2. The toluene and heptane are distilled out on a rotary evaporator at ≦40° C. under reduced pressure. Once most of the volatiles are removed, full vacuum (ca. 5 mmHg) is applied and the bath temperature is gradually raised to 85° C. to distill the benzyl alcohol. When the product solution is concentrated to a total weight of 1.322 kg, distillation ceased. The product mixture is then dissolved in 2/1 hexane/EtOAc (ca. 1.5 mL/g of crude material), applied to a 4.5 kg column of 200-400 mesh silica gel, and gravity eluted with the same solvent. After a 3 L forerun, fractions are collected. The first 4 fractions are 1.6 L, and the others are about 0.9 L. Fractions 6-20 are combined and evaporated at ˜45° C. under reduced pressure to give 1.195 kg of crude 4 as a yellowish-brown oil, ready for use in the next step. ¹H NMR (400 MHz, CDCl₃) δ 7.33 (m, 10H), 5.01 (br s, 2H), 4.57 (s, 1H), 4.34 (s, 1H), 2.62 (dd, J=8.8, 4.8 Hz, 1H), 2.31 (m, 1H), 1.79 (br s, 2H), 1.65 (dd, J=12.4, 8.8 Hz, 1H), 1.46 (m, 2H); ¹³C NMR 100 MHz, CDCl₃) δ 173.4, 155.3, 136.9, 136.1, 128.8, 128.7, 128.5, 128.4, 128.2, 128.1, 67.1, 67.0, 59.6, 56.2, 47.8, 33.7, 29.7, 29.1; MS (GC-MS) m/z (rel. intensity) 365 (M⁺, 2), 258 (1), 230 (13), 186 (7), 168 (25), 91 (100).

Synthesis of 6 via 5a:

Ester 4 (25.0 g, 68.4 mmol) is charged to a 500 mL round bottomed flask equipped with a reflux condenser. Isopropanol (250 mL) is added, and the mixture warmed to 30° C. to effect dissolution. An aqueous solution of NaOH (30%, 13.5 g, 102.6 mmol) is added, and the resulting mixture is heated to 80° C. and held at that temperature for 1 h. Analysis of an aliquot indicated complete conversion to product. The mixture is cooled, and the solvent evaporated under reduced pressure. The residue is partitioned between EtOAc and water. The organic layer is discarded. The aqueous phase containing the sodium salt of carboxylic acid 5a is acidified to pH 2 by adding 3 M HCl. The product is then extracted into EtOAc. Evaporation of the solvent left an oil (16.3 g, 87% yield) that slowly solidifies upon standing.

The crude benzyl carbamate 5a (16.3 g) is dissolved in EtOH (250 mL) and placed in a Fisher-Porter bottle. (BOC)₂O (16.8 g, 76.97 mmol) and Pd/C (Degussa, 50% water w/w, 1.7 g), preferably 5% Pd/C, are added, and the mixture is flushed several times with N₂, and then with H₂ gas. The bottle is then pressurized to ca. 30 psi with H₂, and the mixture is stirred magnetically at rt. Periodically, the CO₂ produced during the reaction is vented, and the system re-charged with fresh H₂. When the reaction is complete (48 h), the mixture is filtered through Celite, and the cake washed well with EtOH. The filtrate and washings are combined and evaporated. The residue is partitioned between dilute aqueous NaOH and EtOAc, and the organic layer is discarded. The basic aqueous phase is then acidified to pH 3 with 2 M HCl to precipitate the product 6. This is collected by suction filtration, washed with water and dried in a vacuum oven at ca. 50° C. The product is obtained as an off-white solid, 10.0 g (61% yield from benzyl ester 4).

Synthesis of 6 via 5b:

Substrate 4 (1.14 kg, 2.31 mol) is dissolved in 2 L of EtOH and transfer to 1-gallon stainless steel autoclave, rinsing it in with a little EtOH. The Pd catalyst (5% Pd/C, 60% water by weight, 115 g) is added as a slurry in EtOH, and the system is flushed several times with N₂ and several times with H₂. Finally, the system is pressurized to ca. 50 psi with H₂. The mixture is stirred at 1000 rpm and maintained overnight at 25° C. and 50 psi H₂ by computer control. The next day, a sample shows that the reaction is not complete. A fresh, 32 g portion of catalyst is added to the reaction mixture through the sample port as a slurry in EtOH. Stirring is continued overnight at 25° C. under 50 psi H₂. The next day, a sample indicats that the reaction is nearly complete. After the normal flushing cycle, the reaction mixture is suctioned out of the autoclave into a 4 L Erlenmeyer flask. The autoclave is rinsed with additional EtOH, and this is also suctioned out and added to the product mixture. The mixture is suction filtered through a bed of Celite, with the cake being washed well with EtOH. A clear, green filtrate is obtained. The EtOH is evaporated under reduced pressure on a rotovap at 60° C. to give a thick, brown gum. This is partitioned between EtOAc (1.5 L) and water (1.5 L) in a 4 L separatory funnel. The EtOAc layer is washed again with water (1 L). The water phases are combined and washed with EtOAc (1 L). The aqueous phase containing the amino acid is then transferred to a distilling flask and the water is distilled out, aided by the periodic addition of iPrOH (to azeotrope the water). When the solution is down to about 0.5 L and estimated to be anhydrous or nearly so, the solution is diluted with 750 mL of EtOAc. This gives rise to the formation of two layers (oiling) rather than precipitation of the solid product. Most of the solvent is therefore evaporated again, and 1 L of EtOAc is added to the residue. Upon mixing, a wet, clumpy solid forms. This becomes more powdery as stirring continues. The suspension is allowed to stand overnight. The lumpy, gray-tan solid is collected on a 2 L, sintered glass filter funnel and washed thoroughly with EtOAc while breaking up the lumps with a large spatula. The solid is dried in air, then in a vacuum oven at 45° C. with a slight air sweep. The dried material for 5b weighs 278 g. ¹H NMR (400 MHz, D₂O) δ4.17 (d, J=2.7 Hz, 1H), 4.11 (d, J=3.0 Hz, 1H), 2.56 (dd, J=9.5, 4.8 Hz, 1H), 1.98 (dd, J=13.3, 9.8 Hz, 1H), 1.92-1.75 (m, 3H), 1.68-1.55 (m, 2H); ¹³C NMR (100 MHz, D₂O) δ 181.0, 62.0, 58.3, 46.9, 33.1, 28.7, 26.2, 25.5.

A 5 L, 4-necked round bottom flask is fitted with a thermocouple, an overhead stirrer, a dropping funnel and an N₂ inlet. Solid substrate 5b (249 g, 1.77 mol) is transferred to the flask, rinsing it in with 50 mL of water. THF (2 L) is added, and the apparatus is immersed in an ice/water bath. The aqueous KOH (25%, 400 g, 1.78 mol) is added at a rate to keep the internal temperature <18° C. A 75% solution of (BOC)₂O in THF (620 g, 2.13 mol) is charged to the dropping funnel. The substrate solution is allowed to cool to 7.2° C., whereupon addition of the (BOC)₂O is started. The addition took 45 min, keeping the reaction mixture <7° C. The mixture is stirred at about 5° C. for 2 h, eventually depositing a small amount of a white precipitate. The mixture is then allowed to warm slowly to rt and stir overnight. HPLC analysis of a sample shows only about 50% conversion. To the mixture is first added another 40 g of 25% KOH, and then is added another 65 g portion of 75% (BOC)₂O in THF over 15 min at rt. The reaction mixture is warmed to about 30° C. for 4 h, during which time gas (CO₂) is seen to exit the mineral oil bubbler. Finally, the mixture is allowed to cool to rt and stir over the weekend, whereupon HPLC analysis of a sample shows >99% conversion to 6. The mixture is transferred portionwise to a 3 L distilling flask, and most of the THF is evaporated on a rotovap at 35-40° C. This gives a white solid with a yellow supernatant (mostly water). More water is added to dissolve all of the salt. To this basic aqueous solution is added 1 L of EtOAc. After mixing and allowing the phases to separate, the aqueous layer is collected and the organic layer is extracted again with ˜0.5% aqueous KOH. The aqueous layers are combined and washed with EtOAc to remove residual neutral impurities. The aqueous solution of the carboxylate salt is then placed on the rotovap and residual organic solvent is evaporated. The solution is then transferred to the 5 L flask and cooled to ˜5° C. The product is precipitated by adding 2 M HCl (900 mL) over 3 h and with vigorous stirring, such that the internal temperature remains below 7° C. The final pH is 3-4. The product is collected by vacuum filtration on a 2 L sintered glass funnel, using 500 mL of water to rinse out the flask. The vacuum is disconnected, and another 500 mL of water is added to the filter cake. The slurry is mixed manually for 5 min, then the water (pH 5) is suctioned through. The solid (mp 174° C., DSC) is pulled dry on the filter overnight, reaching a constant weight of 322.6 g (76% yield). ¹H NMR (400 MHz, CDCl₃) δ 4.54 (d, J=4.0 Hz, 1H), 4.29 (d, J=4.4 Hz, 1H), 2.57 (dd, J=8.8, 4.8 Hz, 1H), 2.22 (m, 1H), 1.79 (m, 2H), 1.61 (dd, J=12.4, 8.8 Hz, 1H), 1.47 (m, 1H), 1.4 (s, 9H); ¹³C NMR (100 MHz, 45° C., CDCl₃) δ 178.4, 155.1, 80.2, 59.5, 56.1, 47.5, 33.5, 29.7, 29.0, 28.4; Anal. Calcd for C₁₂H₁₉NO₄: C, 59.73; H, 7.94; N, 5.81. Found: C, 59.67; H, 8.08; N, 5.75.

Curtius Rearrangement:

A 4-necked, 5 L round bottom flask under N₂ is equipped with a reflux condenser, a dropping funnel, a thermocouple, and an overhead stirrer, and is placed in a heating mantle. Solid 6 (335 g, 1.39 mol) is added to the flask, followed by 2 L of toluene. The resulting suspension is stirred at rt while the triethylamine (207 mL, 1.46 mol) is added. The solid rapidly dissolves to give a brown solution. Diphenyl phosphoryl azide (DPPA, 394 g, 1.39 mol) is dissolved in 400 mL of toluene, and transferred to the dropping funnel with a 100 mL toluene rinse. The substrate/Et₃N solution in the 5 L flask is heated to 50° C. over about 1 h, then slow addition of the DPPA solution is started. The reagent is added in at a rate to maintain the reaction temperature between 60 and 70° C. Nitrogen release is easily controlled at all times. The total addition time is 2 h 20 min. After the addition is complete, residual reagent is rinsed in with a little toluene, and the mixture is then heated slowly to 75° C. over 2 h. By this time, N₂ evolution had essentially ceased. Neat benzyl alcohol (152 mL, 1.46 mol) is placed in the addition funnel, and added in at a rate to keep the reaction temperature between 75 and 80° C. The addition is complete in 35 min, then the heating mantle is connected to a temperature controller set to 80° C., and the mixture is heated for a total of 20 h at 80° C. After the heating period is over, the mantle is removed and the mixture is allowed to cool to 30° C. over 2.5 h. The mixture is then transferred to a separatory funnel, and washed with a total of 2 kg of aqueous NaHCO₃, in two portions. The washes are combined and back extracted with 500 mL of toluene. The organic layers are combined and dried over Na₂SO₄. The bulk of the product solution is decanted into a distilling flask and concentrated on a rotovap at 45° C. The last of the product solution and a toluene rinse of the Na₂SO₄ are filtered into the distilling flask and concentrated similarly. A large, sintered glass filter funnel is charged with 2 kg of silica gel (230-400 mesh). A 40% solution of EtOAc in hexane is prepared and pulled through the silica under vacuum to pack it. The crude Curtius rearrangement product is dissolved in 60/40 hexane/EtOAc and applied to the silica. The product is eluted by gravity, with 1.5 L fractions being collected. Fractions 3-9 are pooled and evaporated. During evaporation, white crystals are seen to form. A small sample of crystals is collected by filtration, washed with 5% EtOAc in hexane, and shown to be nearly pure desired product by NMR. The bulk suspension is evaporated to dryness, and placed on a high vacuum line overnight. A beige solid is obtained (444 g) for 7 still containing some residual benzyl alcohol. ¹H NMR (400 MHz, CDCl₃) δ 7.34 (m, 5H), 5.08 (s, 2H), 4.22 (t, J=4.8 Hz, 1H), 4.12 (m, 1H), 4.11 (m, 1H), 3.77 (ddd, J=16.0, 8.0, 3.3 Hz, 1H), 1.92 (dd, J=12.8, 8.0 Hz, 1H), 1.77 to 1.63 (m, 3H), 1.51-1.30 (m, 12H); ¹³C NMR (100 MHz, CD₃OD) δ 157.0, 156.3, 137.1, 128.2, 127.8, 127.6, 80.0, 66.2, 61.4, 55.3, 55.0, 37.8, 28.1, 27.4, 25.4.

Synthesis of 6 from Tropinone:

Preparation of BOC Nortropinone

Triphosgene (64.0 g, 0.216 mol, 0.6 eq) is dissolved in toluene (200 mL) at rt in a 1 L jacketed reactor under N₂. The apparatus is equipped with a reflux condenser, an overhead stirrer, a thermocouple and a dropping funnel. A vent line is attached so that gases produced during the reaction are directed to a scrubber containing aqueous NaOH and ethylene glycol. Tropinone (50.0 g, 0.36 mol, 1 eq) is dissolved separately in toluene (100 mL) and added to the triphosgene solution over 15 min. The mixture is then warmed to 35-40° C. and held at that temperature until analysis of aliquots indicated that no further conversion is taking place (16 h). The mixture is cooled to rt, treated with water (200 mL), and is stirred vigorously to mix the phases. Hydrolysis of the carbamoyl chloride started spontaneously with vigorous gas evolution accompanied by a small exotherm, and is driven to completion by gentle warming at 40° C. for 1 h. The mixture is then cooled to rt and the phases are allowed to separate and settle. The aqueous phase containing the hydrochloride of 12 is transferred to a separate vessel, brought to pH 12 by addition of aqueous NaOH, and covered with a layer of fresh toluene. BOC anhydride (78.4 g, 0.36 mol, 1 eq) is added via dropping funnel over 30 min and the mixture is stirred vigorously at rt. Due to the formation of CO₂ as the reaction progressed, the pH of the aqueous phase dropped. Aqueous NaOH is added periodically to bring the mixture back to pH 11-12. When the reaction is complete, a small amount of 4-dimethylaminopyridine (DMAP) is added to catalyze the destruction of residual (BOC)₂O. When this is complete, stirring is stopped and the phases are allowed to separate and settle. The aqueous phase is drained, and the organic phase containing 12 is washed sequentially with dilute aqueous HOAc, aqueous NaHCO₃ and brine. The product solution is dried (Na₂SO₄), filtered and evaporated to leave the product as an off-white solid (69.5 g, 86% from tropinone).

Silylation/Chlorination/Favorskii Rearrangement

12 (17.0 g, 75.56 mmol) is dissolved in 170 mL of toluene in a 1 L jacketed reactor under N₂. The solution is maintained at 20° C., and neat DBU (15.8 mL, 105.8 mmol, 1.4 eq) is added via syringe. Immediately thereafter, neat TMS-Cl (12.4 mL, 98.22 mmol, 1.3 eq) is added via syringe over 5 min. A precipitate of DBU hydrochloride forms rapidly. The mixture is stirred at 30° C. for 6 h, and then allowed to cool to rt and stirred overnight. A ca. 99% conversion to the silyl enol ether is obtained. The mixture is treated with 150 mL of ice-cold water and agitated vigorously for 5 min to mix the phases. After allowing the phases to separate and settle, the water layer is drained and the organic phase washed similarly with brine. The product solution is dried (Na₂SO₄), and filtered into a distilling flask. Some of the toluene is evaporated at rt under reduced pressure to further dry the product solution. The solution is then diluted with 50 mL of EtOAc, cooled to ca. 0-5° C. in an ice bath, and treated with solid TCCA (5.85 g, 25.16 mmol, 0.33 eq) in one portion. The mixture is stirred at 0° C. for 1 h, whereupon the starting silyl enol ether is completely consumed. The mixture is warmed to rt, diluted with more EtOAc (100 mL), poured into water and agitated. After allowing the phases to separate and settle, the aqueous phase is drained. The organic phase is concentrated under reduced pressure at 35° C. to remove most of the EtOAc and toluene. The residue is diluted with 100 mL of EtOH and concentrated again to about 75 mL total volume to give tert-butyl 2-chloro-3-[(trimethylsilyl)oxy]-8-azabicyclo[3.2.1]oct-2-ene-8-carboxylate.

The crude chlorination mixture from above is diluted with EtOH (100 mL) and placed in a flask in an ice-water bath. The mixture is treated with 25% aqueous NaOH (36 g, 226.7 mmol), and is stirred for 2 h, whereupon the starting material is completely consumed. The mixture is poured into water and extracted twice with MTBE. The organic phases are combined and retained for possible later analysis. The basic aqueous phase containing the sodium salt of the Favorskii product is cooled to less than 10° C. and carefully brought to pH to 3 with 2 M HCl. The resulting precipitate is collected by vacuum filtration and washed with water until the washings are nearly neutral pH. After air-drying, the solid (mp 174° C., DSC) reached a constant weight of 8.1 g. The filtrate from the acidification was extracted with MTBE. The organic phase was washed with water and brine, dried (Na₂SO₄), filtered and evaporated to get additional product. The total yield was 8.9 g (49%). ¹H NMR (CDCl₃) δ 4.54 (d, J=4.0 Hz, 1H), 4.29 (d, J=4.4 Hz, 1H), 2.57 (dd, J=8.8, 4.8 Hz, 1H), 2.22 (m, 1H), 1.79 (m, 2H), 1.61 (dd, J=12.4, 8.8 Hz, 1H), 1.47 (m, 1H), 1.4 (s, 9H); ¹³C NMR (45° C., CDCl₃) δ 178.4, 155.1, 80.2, 59.5, 56.1, 47.5, 33.5, 29.7, 29.0, 28.4; Anal. Calcd for C₁₂H₁₉NO₄: C, 59.73; H, 7.94; N, 5.81. Found: C, 59.67; H, 8.08; N, 5.75.

Chromatographic Resolution of the Enantiomers:

### EXAMPLE 1

| Operating Parameters | |
|---|---|
| Column (CSP): | Daicel Chiralcel OD, 20 micron |
| Mobile Phase: | 20/80 (v/v) isopropanol/heptane |
| Column Length: | 21 cm |
| Column I.D.: | 8 cm |
| Feed Injection: | ˜40 mL of 50 mg/ml solution |
| | (2 g of racemate) |
| Eluent Flowrate: | 300 mL/min. |
| Temperature: | ambient |
| Separation performance | |
| 8 purity (%) | 100.0% |
| 8 yield | 79% |
| Other enantiomer purity (%) | 97.2% |
| Productivity (kg racemate/kg CSP/day) | 0.67 |
| Solvent Consumption (L/g racemate) | 0.84 |

### EXAMPLE 2

| Operating Parameters | |
|---|---|
| Column (CSP): | Daicel Chiralcel OD, 20 |
| | micron |
| Mobile Phase: | 20/80 (v/v) isopropanol/ |
| | heptane |
| Column Length: | 9.0 cm |
| Column I.D.: | 4.80 cm |
| Number of Columns: | 8 columns |
| Zones: | 2-2-2-2 |
| Feed Concentration: | 45 g racemate/L mobile phase |
| Eluent Flowrate: | 129.89 mL/minute |
| Feed Flowrate: | 32.43 mL/minute |
| Extract Flowrate: | 110.73 mL/minute |
| Raffinate Flowrate: | 25.59 mL/minute |
| Period: | 0.89 minute |
| Temperature: | ambient |
| SMB performance | |
| 8 purity (%) | 100% |
| 8 recovery yield (%) | 98.3% |
| Productivity (kg enantiomer/kg CSP/day) | 2.50 |
| Solvent Consumption (L/g racemate) | 0.084 |

Hydrogenolysis of 8:

Substrate 8 (189 g, 0.546 mol) and Pd catalyst (5% Pd/C, 60% water by weight, 18.8 g) are placed in a 2 L Parr bottle and 1 L of anhydrous EtOH is added. An additional 0.2 L of EtOH is used to rinse in the substrate. The mixture is swirled to dissolve most of the substrate, then placed on the Parr shaker. The system is flushed 3 times with N₂, with brief shaking during each flush. The system is similarly flushed 3 times with H₂, and then pressurized to 60 psi with H₂. Shaking is started with the reaction mixture at 24° C. (internal temperature). Hydrogen uptake is very rapid, and the reaction temperature rises from 24° C. to 32.7° C. during the course of the reaction. About every 5 min, when the pressure drops to about 20 psi, the system is flushed with H₂ and re-charged to 60 psi. This is done 3 times before hydrogen uptake and the exotherm abruptly cease. The total shaking time to this point is 22 min. The shaking is continued for 15 min, with no further uptake of H₂, then a sample is taken and the system re-charged to 50 psi with H₂ and shaken while the sample is analyzed. Analysis of the sample by ¹H NMR shows complete conversion of 8 to 1. The Parr bottle is removed from the shaker and allowed to vent residual H₂ gas for several hours. The reaction mixture is then filtered through a medium sintered glass filter without the use of Celite or any other filter aid. A small amount of very fine catalyst particles pass through this filter. The filtrate and subsequent cake washings are combined and evaporated down to a volume of about 800 mL. This is then filtered through a fine sintered glass filter, again without the use of any filter aid. A clear, very pale yellow solution is obtained. Evaporation under reduced pressure left a pale yellow slush. This is further dried on a high vacuum line until NMR analysis shows the residual EtOH content to be about 0.2% by weight. The material requires no other purification. The product is obtained in quantitative yield (115 g) from 8. ¹H NMR (400 MHz, CDCl₃) δ 4.15 (br s, 1H), 3.85 (br s, 1H), 2.91 (dd, J=7.7, 3.0 Hz, 1H), 1.76 (dd, J=12.8, 7.8 Hz, 1H), 1.68 (br s, 2H), 1.65-1.54 (m, 2H), 1.41 (s, 9H), 1.34-1.20 (m, 3H); ¹³C NMR (100 MHz, 45° C., CDCl₃) δ 155.3, 79.4, 64.3, 55.6, 55.4, 41.9, 28.20, 28.16, 25.8.

1. The process comprising preparing dibenzyl 7-azabicyclo[2.2.1]heptane-2,7-dicarboxylate (4) from phenyl 2-bromo-3-oxo-8-azabicyclo[3.2.1]octane-8-carboxylate (3), comprising dissolving 3 in water or an organic solvent, optionally adding 3 as a ca. 50% w/v solution in a suitable solvent, optionally toluene,
further comprising adding a base,
further optionally comprising conducting the reaction at a temperature from about −10° C. to about 50° C., and
further comprising isolating 4, optionally having stereochemical purity of exo versus endo ratio being about 100:1.

2. The process of claim 1, wherein the organic solvent includes toluene, acetonitrile, dimethoxyethane, diethyl ether, methanol, ethanol, isopropanol, or benzyl alcohol.

3. The process of claim 2, wherein the organic solvent is benzyl alcohol.

4. The process of claim 2, wherein from about 1 eq to about 3 eq of base are used.

5. The process of claim 4, wherein the base includes sodium or potassium hydroxide, or the sodium or potassium salts of the alcoholic solvent.

6. The process of claim 5, wherein 2.2 eq of this base are used.

7. The process of claim 5, wherein the base is sodium benzyloxide.

8. The process of claim 5, wherein the reaction is conducted at a concentration from about 3 mL to about 10 mL of solvent per gram of 3.

9. The process of claim 8, wherein about 4 mL of solvent are used per gram of 3.

10. The process of claim 8, wherein the reaction is conducted at about 5° C.

11. The process of claim 8, wherein the reaction is allowed to stir for about 1 hour or until less than 1% of bromoketone 3 or the corresponding benzyl carbamate remains.

12. The process of claim 11, wherein 4 is isolated further comprising diluting the reaction mixture with 1:1 heptane/toluene;
further comprising neutralizing with conc. HCl;
further comprising extracting the organic phase with aqueous NaOH;
further comprising washing with brine, drying over a drying agent, filtering, and concentrating using standard procedures; and
further comprising removal of excess benzyl alcohol.

13. The process of claim 12, wherein the drying agent is Na₂SO₄.

14. The process of claim 12, wherein the concentrating occurs under reduced pressure at low temperature.

15. The process of claim 14, wherein the temperature is about 40° C.

16. The process of claim 12, wherein the benzyl alcohol is removed by distillation.

17. The process of claim 16, wherein the distillation occurs at about 5 mmHg with a bath temperature of up to 90° C.

18. The process of claim 12, wherein 4 is obtained with as a ratio of about 100:1 that is exo/endo.

19. The process of claim 1, further comprising preparing 3 from phenyl 3-oxo-8-azabicyclo[3.2.1]octane-8-carboxylate (2), comprising dissolving 2 in an inert organic solvent,
further comprising heating the reaction,
further comprising adding anhydrous CuBr₂,
further comprising isolating 3.

20. The process of claim 19, wherein the inert organic solvent includes toluene, acetonitrile, chloroform, an ethyl acetate/chloroform mixture, or ethyl acetate.

21. The process of claim 20, wherein the solvent is ethyl acetate.

22. The process of claim 19, wherein the amount of solvent used is from about 5 mL to about 20 mL of solvent per gram of 2.

23. The process of claim 22, wherein the amount of solvent used is about 10 mL per gram of 2.

24. The process of claim 19, wherein the reaction is heated from about 50° C. to about 110° C.

25. The process of claim 24, wherein the reaction is conducted by refluxing in EtOAc.

26. The process of claim 19, wherein from about 1.9 to about 2.1 eq of CuBr₂ are used.

27. The process of claim 26, wherein 2.0 eq are used.

28. The process of claim 19, wherein the reaction is allowed to stir until 2 is less than 1%.

29. The process of claim 19, wherein 3 is isolated using 1 or more suitable solvents, optionally a solution of toluene being from about 25 to about 75% w/v;
further comprising filtering off the CuBr;
further comprising washing the product solution with water and aqueous NaHCO₃, optionally 5%, removing the EtOAc; and
further comprising adding suitable solvent to give the desired concentration.

30. The process of claim 19, further comprising preparing 2 from tropinone, comprising dissolving tropinone in an inert organic reaction solvent,
further comprising adding a weak insoluble inorganic base,
further comprising conducting the reaction at a temperature of at least about 0° C.,
further comprising adding phenyl chloroformate, optionally heating to reflux after addition is complete,
further comprising isolating 2.

31. The process of claim 30, wherein the inert organic solvent includes toluene, acetonitrile, dichloromethane, or ethyl acetate.

32. The process of claim 30, wherein the weak insoluble inorganic base includes sodium or potassium bicarbonate or sodium or potassium carbonate.

33. The process of claim 30, wherein the reaction is heating from about 0° C. to about 110° C.

34. The process of claim 30, wherein the chloroformate is added at a rate to keep the reaction temperature less than about 30° C.

35. The process of claim 30, wherein 2 is isolated comprising slowly adding an antisolvent;
further comprising cooling the reaction from about −10° C. to about 20° C.;
further comprising removing 2 by filtration, washing with a dilute acid solution;
further comprising washing with water; and
optionally further comprising washing with a dilute base solution.

36. The process of claim 35, wherein the antisolvent is hexane or heptane.

37. The process of claim 35, wherein an amount of antisolvent is used to give a concentration of about 1 mL to about 5 mL of antisolvent per mL of reaction solvent.

38. The process of claim 37, wherein about 2 mL of antisolvent per mL of solvent are used.

39. The process of claim 35, wherein the dilute acid solution is from about 0.5% to about 5%.

40. The process of claim 39, wherein the dilute acid solution includes sulfuric, phosphoric, or hydrochloric acid.

41. The process of claim 35, wherein the optional dilute base solution is from about 0.5% to about 5%.

42. The process of claim 35, wherein 2 is obtained with about 99% purity.

43. The process of claim 30, further comprising the preparation of 7-(tert-butoxycarbonyl)-7-azabicyclo[2.2.1]heptane-2-carboxylic acid (6) from 4, comprising dissolving 4 in a low molecular weight alcohol, optionally ethanol or isopropanol;
further comprising using Pd/C, optionally 5-10% Pd/C and optionally from about 0.5 to about 5 g of Pd/C per gram of 4;
further comprising applying hydrogen, optionally from about 30 psi to about 60 psi and further optionally at a temperature of at least room temperature;
further comprising isolating 7-azabicyclo[2.2.1]heptane-2-carboxylic acid (5b);
further comprising dissolving 5b in THF and acqueous KOH, optionally 10% aqueous, to give a homogeneous solution, optionally adding aqueous KOH more than once;
further comprising adding (BOC)₂O, optionally adding (BOC)₂O more than once; and
further comprising isolating 6.

44. The process of claim 43, further comprising preparing exo-tert-butyl 2(R(+)-{[(benzyloxy)carbonyl]amino}-7-azabicyclo[2.2.1]heptane-7-carboxylate (8) from 6, by resolving (2R)-7-(tert-butoxycarbonyl)-7-azabicyclo[2.2.1]heptane-2-carboxylic acid (15) from 6;
further comprising dissolving 15 and a non-nucleophilic organic soluble base, optionally Et₃N and optionally from about 1 eq to about 1.5 eq, in toluene;
further comprising heating the solution, optionally from about 30° C. to about 80° C.;
further comprising adding DPPA, optionally from about 0.95 eq to about 1.2 eq, and further optionally at a rate to control the reaction temperature and nitrogen off-gassing rate;
further comprising heating the reaction, optionally from about 30° C. to 110° C., once the temperature and nitrogen off gassing begins to fall;
further comprising adding benzyl alcohol, optionally from about 0.95 eq to about 1.5 eq;
further comprising stirring the reaction, optionally at a temperature from about 30° C. to 110° C. and optionally for about 1 hour or until residual 15 is less than 1% relative to 8;
further comprising a mildly basic aqueous work-up, optionally purifying using silica gel chromatography eluting with EtOAc, optionally 40-50% EtOAc in hexane; and
further comprising isolating 8.

45. The process of claim 44, further comprising preparing exo-(t-butyl 2R(+))-2-amino-7-azabicyclo[2.2.1]heptane-7-carboxylate (1) from 8 by hydrogenolysis.

46. The process of claim 43, further comprising preparing exo-tert-butyl 2(R(+)-{[(benzyloxy)carbonyl]amino}-7-azabicyclo[2.2.1]heptane-7-carboxylate (8) from tert-butyl 2-{[(benzyloxy)carbonyl]amino}-7-azabicyclo[2.2.1]heptane-7-carboxylate (7) after preparing 7 from 6;
further comprising dissolving 6 and a non-nucleophilic organic soluble base, optionally from about 1 to about 1.5 equivalents relative to substrate 6 and optionally using triethylamine or diisopropyl ethylamine as the base, in a high-boiling, inert organic solvent, optionally toluene or xylene and optionally to give a resulting concentration of solvent to substrate of about 3 mL to about 10 mL of solvent per gram of substrate;
further comprising heating the solution, optionally from about 30° C. to about 80° C.;
further comprising adding DPPA, optionally from about 0.95 to about 1.2 equivalents and optionally at a rate to control the reaction temperature and nitrogen off-gassing rate;
further comprising heating the reaction, optionally from about 30° C. to about 110° C. once the temperature and nitrogen off gassing begins to fall;
further comprising adding benzyl alcohol, optionally from about 0.95 to about 1.5 eq, and heating the reaction, optionally at from about 30° C. to 110° C. and optionally until residual 6 is less than 1% relative to 7 or about 1 hour after addition of benzyl alcohol;
further comprising a mildly basic aqueous work-up, optionally NaHCO₃;
further comprising isolating 7;
further comprising crystallizing 7 from hexane/ethyl acetate; and
further comprising resolving 8 from 7.

47. The process of claim 46, further comprising preparing exo-(t-butyl 2R(+))-2-amino-7-azabicyclo[2.2.1]heptane-7-carboxylate (1) from 8 by hydrogenolysis.

48. The process of claim 1, further comprising the preparation of 7-(tert-butoxycarbonyl)-7-azabicyclo[2.2.1]heptane-2-carboxylic acid (6) from 4, comprising dissolving 4 in a low molecular weight alcohol, optionally ethanol or isopropanol;
further comprising using Pd/C, optionally 5-10% Pd/C and optionally from about 0.5 to about 5 g of Pd/C per gram of 4;
further comprising applying hydrogen, optionally from about 30 psi to about 60 psi and further optionally at a temperature of at least room temperature;
further comprising isolating 7-azabicyclo[2.2.1]heptane-2-carboxylic acid (5b);
further comprising dissolving 5b in THF and acqueous KOH, optionally 10% aqueous, to give a homogeneous solution, optionally adding aqueous KOH more than once;
further comprising adding (BOC)₂O, optionally adding (BOC)₂O more than once; and
further comprising isolating 6.

49. The process of claim 48, further comprising preparing exo-tert-butyl 2(R(+)-{[(benzyloxy)carbonyl]amino}-7-azabicyclo[2.2.1]heptane-7-carboxylate (8) from 6, by resolving (2R)-7-(tert-butoxycarbonyl)-7-azabicyclo[2.2.1]heptane-2-carboxylic acid (15) from 6;
further comprising dissolving 15 and a non-nucleophilic organic soluble base, optionally Et₃N and optionally from about 1 eq to about 1.5 eq, in toluene;
further comprising heating the solution, optionally from about 30° C. to about 80° C.;
further comprising adding DPPA, optionally from about 0.95 eq to about 1.2 eq, and further optionally at a rate to control the reaction temperature and nitrogen off-gassing rate;
further comprising heating the reaction, optionally from about 30° C. to 110° C., once the temperature and nitrogen off gassing begins to fall;
further comprising adding benzyl alcohol, optionally from about 0.95 eq to about 1.5 eq;
further comprising stirring the reaction, optionally at a temperature from about 30° C. to 110° C., for about 1 hour or until residual 15 is less than 1% relative to 8;
further comprising a mildly basic aqueous work-up, optionally purifying using silica gel chromatography eluting with EtOAc, optionally 40-50% EtOAc in hexane; and
further comprising isolating 8.

50. The process of claim 49, further comprising preparing exo-(t-butyl 2R(+))-2-amino-7-azabicyclo[2.2.1]heptane-7-carboxylate (1) from 8 by hydrogenolysis.

51. The process of claim 48, further comprising preparing exo-tert-butyl 2(R(+)-{[(benzyloxy)carbonyl]amino}-7-azabicyclo[2.2.1]heptane-7-carboxylate (8) from tert-butyl 2-{[(benzyloxy)carbonyl]amino}-7-azabicyclo[2.2.1]heptane-7-carboxylate (7) after preparing 7 from 6;
further comprising dissolving 6 and a non-nucleophilic organic soluble base, optionally from about 1 to about 1.5 equivalents relative to substrate 6 and optionally using triethylamine or diisopropyl ethylamine as the base, in a high-boiling, inert organic solvent, optionally toluene or xylene and optionally to give a resulting concentration of solvent to substrate of about 3 mL to about 10 mL of solvent per gram of substrate;
further comprising heating the solution, optionally from about 30° C. to about 80° C.;
further comprising adding DPPA, optionally from about 0.95 to about 1.2 equivalents and optionally at a rate to control the reaction temperature and nitrogen off-gassing rate;
further comprising heating the reaction from about 30° C. to about 110° C. once the temperature and nitrogen off gassing begins to fall;
further comprising adding benzyl alcohol, optionally from about 0.95 to about 1.5 eq, and heating the reaction at from about 30° C. to 110° C., until residual 6 is less than 1% relative to 7 or about 1 hour after addition of benzyl alcohol;
further comprising a mildly basic aqueous work-up, optionally NaHCO₃;
further comprising isolating 7;
further comprising crystallizing 7 from hexane/ethyl acetate; and
further comprising resolving 8 from 7.

52. The process of claim 51, further comprising preparing exo-(t-butyl 2R(+))-2-amino-7-azabicyclo[2.2.1]heptane-7-carboxylate (1) from 8 by hydrogenolysis.

53. The process of claim 1 further comprising preparing 7-(tert-butoxycarbonyl)-7-azabicyclo[2.2.1]heptane-2-carboxylic acid (6) from tert-butyl 3-oxo-8-azabicyclo[3.2.1]octane-8-carboxylate (12), comprising treating 12 with an amine base, wherein the base is optionally diazabicycloundecene or DBU, and TMS-Cl to give a silyl enol ether intermediate;
further comprising treating the enol intermediate in EtOAc with TCCA to give a chlorinated intermediate;
further optionally comprising isolating the chlorinated intermediate and optionally removing EtOAc and toluene; and
further comprising treating the chlorinated intermediate with a base including KOH in an alcoholic solvent, optionally isopropanol, or NaOH in an alcoholic solvent, optionally ethanol.

54. The process of claim 53, further comprising preparing 12 from tropinone, comprising addition of triphosgene to tropinone in an inert solvent, optionally toluene;
further comprising addition of water;
further comprising adding an aqueous base, optionally NaOH, to raise the reaction pH, optionally above 10;
further optionally comprising the addition of more toluene; and
further comprising addition of (BOC)₂O, optionally comprising the addition of DMAP to catalyze the destruction of residual (BOC)₂O and isolation of 12.

55. The process of claim 54, further comprising addition of neat DBU, optionally 1.4 eq, and followed by the addition of neat TMS-Cl, optionally 1.3 eq, to 12 in toluene in that order;
further comprising isolating the enol intermediate;
further comprising the addition of solid TCCA to the enol intermediate in EtOAc optionally cooled to about 0-5° C., and further optionally stirring at 0° C. until the enol intermediate is consumed; and
further comprising isolating the chlorinated intermediate and removing EtOAc and toluene.

56. The process of claim 55, further comprising preparing exo-tert-butyl 2(R(+)-{[(benzyloxy)carbonyl]amino}-7-azabicyclo[2.2.1]heptane-7-carboxylate (8) from 6, by resolving (2R)-7-(tert-butoxycarbonyl)-7-azabicyclo[2.2.1]heptane-2-carboxylic acid (15) from 6;
further comprising dissolving 15 and a non-nucleophilic organic soluble base, optionally Et₃N and optionally from about 1 eq to about 1.5 eq, in toluene;
further comprising heating the solution, optionally from about 30° C. to about 80° C.;
further comprising adding DPPA, optionally from about 0.95 eq to about 1.2 eq, and further optionally at a rate to control the reaction temperature and nitrogen off-gassing rate;
further comprising heating the reaction, optionally from about 30° C. to 110° C., once the temperature and nitrogen off gassing begins to fall;
further comprising adding benzyl alcohol, optionally from about 0.95 eq to about 1.5 eq;
further comprising stirring the reaction, optionally at a temperature from about 30° C. to 110° C. and optionally for about 1 hour or until residual 15 is less than 1% relative to 8;
further comprising a mildly basic aqueous work-up, optionally purifying using silica gel chromatography eluting with EtOAc, optionally 40-50% EtOAc in hexane; and
further comprising isolating 8.

57. The process of claim 56, further comprising preparing exo-(t-butyl 2R(+))-2-amino-7-azabicyclo[2.2.1]heptane-7-carboxylate (1) from 8 by hydrogenolysis.

58. The process of claim 55, further comprising preparing exo-tert-butyl 2(R(+)-{[(benzyloxy)carbonyl]amino}-7-azabicyclo[2.2.1]heptane-7-carboxylate (8) from tert-butyl 2-{[(benzyloxy)carbonyl]amino}-7-azabicyclo[2.2.1]heptane-7-carboxylate (7) after preparing 7 from 6;
further comprising dissolving 6 and a non-nucleophilic organic soluble base, optionally from about 1 to about 1.5 equivalents relative to substrate 6 and optionally using triethylamine or diisopropyl ethylamine as the base, in a high-boiling, inert organic solvent, optionally toluene or xylene and optionally to give a resulting concentration of solvent to substrate of about 3 mL to about 10 mL of solvent per gram of substrate;
further comprising heating the solution, optionally from about 30° C. to about 80° C.;
further comprising adding DPPA, optionally from about 0.95 to about 1.2 equivalents and optionally at a rate to control the reaction temperature and nitrogen off-gassing rate;
further comprising heating the reaction from about 30° C. to about 110° C. once the temperature and nitrogen off gassing begins to fall;
further comprising adding benzyl alcohol, optionally from about 0.95 to about 1.5 eq, and heating the reaction at from about 30° C. to 110° C., until residual 6 is less than 1% relative to 7 or about 1 hour after addition of benzyl alcohol;
further comprising a mildly basic aqueous work-up, optionally NaHCO₃;
further comprising isolating 7;
further comprising crystallizing 7 from hexane/ethyl acetate; and
further comprising resolving 8 from 7.

59. The process of claim 58, further comprising preparing exo-(t-butyl 2R(+))-2-amino-7-azabicyclo[2.2.1]heptane-7-carboxylate (1) from 8 by hydrogenolysis.

60. A compound that is
exo-(tert-butyl 2R(+))-2-amino-7-azabicyclo[2.2.1]heptane-7-carboxylate (1);
exo-tert-butyl 2(R(+)-{[(benzyloxy)carbonyl]amino}-7-azabicyclo[2.2.1]heptane-7-carboxylate (8);
exo-tert-butyl 2-[(benzyloxy)carbonyl]amino-7-azabicyclo[2.2.1]heptane-7-carboxylate (7);
(2R)-7-(tert-butoxycarbonyl)-7-azabicyclo[2.2.1]heptane-2-carboxylic acid (15); or
7-(tert-butoxycarbonyl)-7-azabicyclo[2.2.1]heptane-2-carboxylic acid (6),
provided that when the compound is an acid, the compound can be an alkali metal or amine salt thereof; further provided that when the compound is an amine, the compound can be an acid salt thereof; and further provided that the compound can have varying degrees of chemical purity and chiral purity.

61. The compound of claim 60 that is exo-(t-butyl 2R(+))-2-amino-7-azabicyclo[2.2.1]heptane-7-carboxylate (1), provided that the compound can be the acid salt thereof; and further provided that the compound can have varying degrees of chemical purity and chiral purity.

62. The compound of claim 61, wherein the compound has at least 95% chemical purity and at least 95% chiral purity.

63. The compound of claim 61, wherein the compound has at least 97% chemical purity and at least 99.5% chiral purity.

64. The compound of claim 60 that is 7-(tert-butoxycarbonyl)-7-azabicyclo[2.2.1]heptane-2-carboxylic acid (6), provided that the compound can be the alkali metal or amine salt thereof, and further provided that the compound can have varying degrees of chemical purity and chiral purity.

65. The compound of claim 64, wherein the compound has at least 95% chemical purity and at least 95% chiral purity.

66. The compound of claim 64, wherein the compound has at least 97% chemical purity and at least 99.5% chiral purity.
